(19) United States
(12) Patent Application Publication
(10) Pub. No.: US 2025/0242057 A1
(43) Pub. Date: Jul. 31, 2025

(54) Disulfide Bond Stabilized Polypeptide Compositions And Methods Of Use

(71) Applicant: Amicus Therapeutics, Inc., Philadelphia, PA (US)

(72) Inventors: Hung V. Do, New Hope, PA (US); Ce Feng Liu, Brooklyn, NY (US)

(73) Assignee: Amicus Therapeutics, Inc., Philadelphia, PA (US)

(21) Appl. No.: 18/808,596

(22) Filed: Aug. 19, 2024

Related U.S. Application Data
(63) Continuation of application No. 17/363,333, filed on Jun. 30, 2021, now Pat. No. 12,064,485.
Application No. 17/363,333 is a division of application No. 16/598,960, filed on Oct. 10, 2019, now Pat. No. 11,097,015.
(60) Provisional application No. 62/744,069, filed on Oct. 10, 2018.

(51) Int. Cl. A61K 48/00; A61K 38/46; A61K 38/47; A61P 43/00; C07H 21/04; C12N 15/79; C12N 15/86

(52) U.S. Cl. CPC A61K 48/0058; A61K 38/465; A61K 38/47; A61K 48/0033; A61P 43/00; C12N 15/86; C07H 21/04; C12N 15/79; C12N 2750/14143; C12N 2800/00; C12Y 301/02022; C12Y 302/01022

(57) ABSTRACT

Provided herein are polypeptides comprising one or more non-native cysteine residues that form a disulfide bridge between non-native cysteines within the protein or between non-native cysteines of two monomers of the protein. Such modified human polypeptides are useful in treatment of genetic diseases via enzyme replacement therapy and/or gene therapy.

### CROSS-REFERENCE

This application is a continuation of U.S. Ser. No. 17/363,333, filed on Jun. 30, 2021, which is a divisional of U.S. Ser. No. 16/598,960, filed on Oct. 10, 2019, now U.S. Pat. No. 11,097,015, which claims the benefit of U.S. Provisional Application No. 62n/44,069, filed Oct. 10, 2018, which applications are hereby incorporated herein by reference in its entirety.

### REFERENCE TO THE SEQUENCE LISTING

The Sequence Listing XML file submitted herewith, identified as “AT18-006-US3.xml” (57,708 bytes, created Apr. 10, 2024), is hereby incorporated by reference.

### BACKGROUND

Genetic diseases can be treated with enzyme replacement therapy using recombinant polypeptides or gene therapy using nucleic acids encoding recombinant proteins. For example, Fabry disease may be treated using recombinant alpha-galactosidase A or small molecule chaperones such as 1-deoxygalactonojirimycin (Migalastat). However, the recombinant wildtype polypeptides often have poor stability at neutral pH and are quickly degraded in serum. This limits the half-life of the therapeutic enzyme substantially, as it is delivered by intravenous infusion.

### SUMMARY

In certain aspects, there are provided gene therapy vectors comprising a nucleic acid construct comprising: a nucleic acid encoding a stabilized form of a protein for treating a genetic disorder. In some embodiments, the stabilized form comprises one or more non-native cysteine residues that form a disulfide bridge between non-native cysteines within the protein or between non-native cysteines of two monomers of the protein. In some embodiments, the protein is selected from the group consisting of alpha-galactosidase A, β-glucocerebrosidase, glucocerebrosidase, lysosomal acid lipase, glycosaminoglycan alpha-L-iduronidase, alpha-L-iduronidase, N-sulfoglucosamine sulfohydrolase (SGSH), N-acetyl-alpha-glucosaminidase (NAGLU), iduronate-2-sulfatase, N-acetylgalactosamine-6-sulfatase, glycosaminoglycan N-acetylgalactosamine 4-sulfatase, alpha-glucosidase, tripeptidyl peptidase 1 (TPP1), palmitoyl protein thioesterases (PPTs), ceroid lipofuscinoses neuronal 1, ceroid lipofuscinoses neuronal 2, ceroid lipofuscinoses neuronal 3, ceroid lipofuscinoses neuronal 4, ceroid lipofuscinoses neuronal 5, ceroid lipofuscinoses neuronal 6, ceroid lipofuscinoses neuronal 7, ceroid lipofuscinoses neuronal 8, ceroid lipofuscinoses neuronal 9, ceroid lipofuscinoses neuronal 10, ceroid lipofuscinoses neuronal 11, ceroid lipofuscinoses neuronal 12, ceroid lipofuscinoses neuronal 13, ceroid lipofuscinoses neuronal 14, ceroid lipofuscinoses neuronal 15, ceroid lipofuscinoses neuronal 16, and cyclin dependent kinase like 5. In some embodiments, the protein is selected from the group consisting of alpha-galactosidase A, β-glucocerebrosidase, glucocerebrosidase, lysosomal acid lipase, glycosaminoglycan alpha-L-iduronidase, alpha-L-iduronidase, N-sulfoglucosamine sulfohydrolase (SGSH), N-acetyl-alpha-glucosaminidase (NAGLU), iduronate-2-sulfatase, N-acetylgalactosamine-6-sulfatase, glycosaminoglycan N-acetylgalactosamine 4-sulfatase, alpha-glucosidase, tripeptidyl peptidase 1 (TPP1), palmitoyl protein thioesterases (PPTs), ceroid lipofuscinoses neuronal 4, ceroid lipofuscinoses neuronal 10 (cathepsin D), ceroid lipofuscinoses neuronal 11 (progranulin), ceroid lipofuscinoses neuronal 13 (cathepsin F), ceroid lipofuscinoses neuronal 14 (KCTD7), ceroid lipofuscinoses neuronal 15 (TBCK), and cyclin dependent kinase like 5. In some embodiments, the stabilized protein comprises a lysosomal enzyme. In some embodiments, the stabilized protein comprises a stabilized α-galactosidase (α-GAL) protein. In some embodiments, the stabilized α-galactosidase A (α-GAL) protein comprises one or more non-native cysteine residues selected from the group consisting of: (i) D233C and I359C; and (ii) M51C and G360C. In some embodiments, the stabilized protein comprises a stabilized palmitoyl protein thioesterase 1 (PPT1). In some embodiments, the stabilized PPT1 protein comprises non-native cysteine residues A171C and A183C. In some embodiments, the stabilized protein has a longer half-life at pH 7.4 compared to a corresponding protein without the non-native cysteines. In some embodiments, the stabilized protein can replace a protein defective or deficient in the genetic disorder. In some embodiments, the stabilized protein can reduce or slow one or more symptoms associated with the genetic disorder. In some embodiments, the stabilized protein is more effective at reducing or slowing one or more symptoms of the genetic disorder, compared to an unstabilized protein. In some embodiments, the genetic disorder is a neurological disorder. In some embodiments, the genetic disorder is a lysosomal storage disorder. In some embodiments, the genetic disorder is selected from the group consisting of aspartylglucosaminuria, Batten disease, cystinosis, Fabry disease, Gaucher disease type I, Gaucher disease type II, Gaucher disease type III, Pompe disease, Tay Sachs disease, Sandhoff disease, metachomatic leukodystrophy, mucolipidosis type I, mucolipidosis type II, mucolipidosis type III, mucolipidosis type IV, Hurler disease, Hunter disease, Sanfilippo disease type A, Sanfilippo disease type B, Sanfilippo disease type C, Sanfilippo disease type D, Morquio disease type A, Morquio disease type B, Maroteau-Lamy disease, Sly disease, Niemann-Pick disease type A, Niemann-Pick disease type B, Niemann-Pick disease type C1, Niemann-Pick disease type C2, Schindler disease type I, Schindler disease type II, adenosine deaminase severe combined immunodeficiency (ADA-SCID), chronic granulomatous disease (CGD), infantile, juvenile and adult forms of neuronal ceroid lipofuscinosis, and CDKL5 deficiency disease. In some embodiments, the gene therapy vector is a viral vector selected from the group consisting of an adenovirus vector, an adeno-associated virus vector, a retrovirus vector, a lentivirus vector, and a herpes virus vector. In some embodiments, the adeno-associated virus is a serotype selected from the group consisting of: AAV-1, AAV-2, AAV-3, AAV-4, AAV-5, AAV-6, AAV-7, AAV-8, AAV-9, AAV-10, AAV-11, AAV-12, AAV-13, AAV rh.74, AAV-B1 and AAV-hu68. In some embodiments, the nucleic acid construct is comprised in a viral vector genome. In some embodiments, the viral vector genome comprises a recombinant AAV (rAAV) genome. In some embodiments, the rAAV genome comprises a self-complementary genome. In some embodiments, the rAAV genome comprises a single-stranded genome. In some embodiments, the rAAV genome comprises a first inverted terminal repeat and a second inverted terminal repeat. In some embodiments, the AAV inverted terminal repeats are AAV2 inverted terminal repeats. In some embodiments, the rAAV genome further comprises an SV40 intron. In some embodiments, the rAAV genome further comprises a poly-adenylation sequence. In some embodiments, the construct further comprises a nucleic acid sequence encoding an α-GAL protein, wherein the nucleic acid sequence is at least 85% identical to one of SEQ ID NOs: 7-12. In some embodiments, the construct further comprises a nucleic acid sequence encoding an α-GAL protein, wherein the α-GAL protein comprises a sequence at least 85% identical to one of SEQ ID NOs: 1-6. In some embodiments, the construct further comprises a nucleic acid sequence encoding an α-GAL protein, wherein the nucleic acid sequence comprises the sequence of one of SEQ ID NOs: 8-12. In some embodiments, the construct further comprises a nucleic acid sequence encoding an α-GAL protein, wherein the α-GAL protein comprises the sequence of one of SEQ ID NOs: 2-6. In some embodiments, the construct further comprises a nucleic acid sequence encoding a PPT1 protein, wherein the nucleic acid sequence is at least 85% identical to one of SEQ ID NOs: 15-16. In some embodiments, the construct further comprises a nucleic acid sequence encoding a PPT1 protein, wherein the PPT1 protein comprises a sequence at least 85% identical to one of SEQ ID NOs: 13-14. In some embodiments, the construct further comprises a nucleic acid sequence encoding a PPT1 protein, wherein the nucleic acid sequence comprises the sequence of SEQ ID NO: 16. In some embodiments, the construct further comprises a nucleic acid sequence encoding a PPT1 protein, wherein the PPT1 protein comprises the sequence of SEQ ID NO: 14. In some embodiments, the construct further comprises a promoter sequence. In some embodiments, the promoter is a constitutive promoter. In some embodiments, the promoter is a tissue-specific promoter. In some embodiments, the construct further comprises one or more nucleic acid sequences selected from the group consisting of: a Kozak sequence, a CrPV IRES, a nucleic acid sequence encoding a linker, a nucleic acid sequence encoding a signal sequence, and a nucleic acid sequence encoding an IGF2 peptide. In some embodiments, the signal peptide sequence comprises a binding immunoglobulin protein (Bip) signal sequence. In some embodiments, the signal peptide sequence comprises the Bip signal sequence comprises an amino acid sequence at least 90% identical to an amino acid sequence selected from the group consisting of SEQ ID NOs: 29-33. In some embodiments, the construct further comprises an internal ribosomal entry sequence (IRES). In some embodiments, the IRES comprises a cricket paralysis virus (CrPV) IRES. In some embodiments, the construct further comprises a nucleic acid sequence encoding a variant IGF2 (vIGF2) peptide. In some embodiments, the vIGF2 peptide comprising an amino acid sequence at least 90% identical to an amino acid sequence selected from the group consisting of SEQ ID NOs: 17-27. In some embodiments, the nucleic acid sequence encoding the vIGF2 peptide is 5′ to the nucleic acid sequence encoding the stabilized form of the protein. In some embodiments, the nucleic acid sequence encoding the vIGF2 peptide is 3′ to the nucleic acid sequence encoding the stabilized form of the protein. In some embodiments, the construct is packaged within a viral capsid.

In additional aspects, there are provided pharmaceutical compositions comprising a gene therapy vector comprising a nucleic acid construct comprising: a nucleic acid encoding a stabilized form of a protein for treating a genetic disorder and a pharmaceutically acceptable excipient, carrier, or diluent. In some embodiments, the stabilized form comprises one or more non-native cysteine residues that form a disulfide bridge between non-native cysteines within the protein or between non-native cysteines of two monomers of the protein. In some embodiments, the protein is selected from the group consisting of alpha-galactosidase A, β-glucocerebrosidase, glucocerebrosidase, lysosomal acid lipase, glycosaminoglycan alpha-L-iduronidase, alpha-L-iduronidase, N-sulfoglucosamine sulfohydrolase (SGSH), N-acetyl-alpha-glucosaminidase (NAGLU), iduronate-2-sulfatase, N-acetylgalactosamine-6-sulfatase, glycosaminoglycan N-acetylgalactosamine 4-sulfatase, alpha-glucosidase, tripeptidyl peptidase 1 (TPP1), palmitoyl protein thioesterases (PPTs), ceroid lipofuscinoses neuronal 1, ceroid lipofuscinoses neuronal 2, ceroid lipofuscinoses neuronal 3, ceroid lipofuscinoses neuronal 4, ceroid lipofuscinoses neuronal 5, ceroid lipofuscinoses neuronal 6, ceroid lipofuscinoses neuronal 7, ceroid lipofuscinoses neuronal 8, ceroid lipofuscinoses neuronal 9, ceroid lipofuscinoses neuronal 10, ceroid lipofuscinoses neuronal 11, ceroid lipofuscinoses neuronal 12, ceroid lipofuscinoses neuronal 13, ceroid lipofuscinoses neuronal 14, ceroid lipofuscinoses neuronal 15, ceroid lipofuscinoses neuronal 16, and cyclin dependent kinase like 5. In some embodiments, the protein is selected from the group consisting of alpha-galactosidase A, β-glucocerebrosidase, glucocerebrosidase, lysosomal acid lipase, glycosaminoglycan alpha-L-iduronidase, alpha-L-iduronidase, N-sulfoglucosamine sulfohydrolase (SGSH), N-acetyl-alpha-glucosaminidase (NAGLU), iduronate-2-sulfatase, N-acetylgalactosamine-6-sulfatase, glycosaminoglycan N-acetylgalactosamine 4-sulfatase, alpha-glucosidase, tripeptidyl peptidase 1 (TPP1), palmitoyl protein thioesterases (PPTs), ceroid lipofuscinoses neuronal 4, ceroid lipofuscinoses neuronal 10 (cathepsin D), ceroid lipofuscinoses neuronal 11 (progranulin), ceroid lipofuscinoses neuronal 13 (cathepsin F), ceroid lipofuscinoses neuronal 14 (KCTD7), ceroid lipofuscinoses neuronal 15 (TBCK), and cyclin dependent kinase like 5. In some embodiments, the stabilized protein comprises a lysosomal enzyme. In some embodiments, the stabilized protein comprises a stabilized α-galactosidase (α-GAL) protein. In some embodiments, the stabilized α-galactosidase A (α-GAL) protein comprises one or more non-native cysteine residues selected from the group consisting of: (i) D233C and I359C; and (ii) M51C and G360C. In some embodiments, the stabilized protein comprises a stabilized palmitoyl protein thioesterase 1 (PPT1). In some embodiments, the stabilized PPT1 protein comprises non-native cysteine residues A171C and A183C. In some embodiments, the stabilized protein has a longer half-life at pH 7.4 compared to a corresponding protein without the non-native cysteines. In some embodiments, the stabilized protein can replace a protein defective or deficient in the genetic disorder. In some embodiments, the stabilized protein can reduce or slow one or more symptoms associated with the genetic disorder. In some embodiments, the stabilized protein is more effective at reducing or slowing one or more symptoms of the genetic disorder, compared to an unstabilized protein. In some embodiments, the genetic disorder is a neurological disorder. In some embodiments, the genetic disorder is a lysosomal storage disorder. In some embodiments, the genetic disorder is selected from the group consisting of aspartylglucosaminuria, Batten disease, cystinosis, Fabry disease, Gaucher disease type I, Gaucher disease type II, Gaucher disease type III, Pompe disease, Tay Sachs disease, Sandhoff disease, metachomatic leukodystrophy, mucolipidosis type I, mucolipidosis type II, mucolipidosis type III, mucolipidosis type IV, Hurler disease, Hunter disease, Sanfilippo disease type A, Sanfilippo disease type B, Sanfilippo disease type C, Sanfilippo disease type D, Morquio disease type A, Morquio disease type B, Maroteau-Lamy disease, Sly disease, Niemann-Pick disease type A, Niemann-Pick disease type B, Niemann-Pick disease type C1, Niemann-Pick disease type C2, Schindler disease type I, Schindler disease type II, adenosine deaminase severe combined immunodeficiency (ADA-SCID), chronic granulomatous disease (CGD), infantile, juvenile and adult forms of neuronal ceroid lipofuscinosis, and CDKL5 deficiency disease. In some embodiments, the gene therapy vector is a viral vector selected from the group consisting of an adenovirus vector, an adeno-associated virus vector, a retrovirus vector, a lentivirus vector, and a herpes virus vector. In some embodiments, the adeno-associated virus is a serotype selected from the group consisting of: AAV-1, AAV-2, AAV-3, AAV-4, AAV-5, AAV-6, AAV-7, AAV-8, AAV-9, AAV-10, AAV-11, AAV-12, AAV-13, AAV rh.74, AAV-B1 and AAV-hu68. In some embodiments, the nucleic acid construct is comprised in a viral vector genome. In some embodiments, the viral vector genome comprises a recombinant AAV (rAAV) genome. In some embodiments, the rAAV genome comprises a self-complementary genome. In some embodiments, the rAAV genome comprises a single-stranded genome. In some embodiments, the rAAV genome comprises a first inverted terminal repeat and a second inverted terminal repeat. In some embodiments, the AAV inverted terminal repeats are AAV2 inverted terminal repeats. In some embodiments, the rAAV genome further comprises an SV40 intron. In some embodiments, the rAAV genome further comprises a poly-adenylation sequence. In some embodiments, the construct further comprises a nucleic acid sequence encoding an α-GAL protein, wherein the nucleic acid sequence is at least 85% identical to one of SEQ ID NOs: 7-12. In some embodiments, the construct further comprises a nucleic acid sequence encoding an α-GAL protein, wherein the α-GAL protein comprises a sequence at least 85% identical to one of SEQ ID NOs: 1-6. In some embodiments, the construct further comprises a nucleic acid sequence encoding an α-GAL protein, wherein the nucleic acid sequence comprises the sequence of one of SEQ ID NOs: 8-12. In some embodiments, the construct further comprises a nucleic acid sequence encoding an α-GAL protein, wherein the α-GAL protein comprises the sequence of one of SEQ ID NOs: 2-6. In some embodiments, the construct further comprises a nucleic acid sequence encoding a PPT1 protein, wherein the nucleic acid sequence is at least 85% identical to one of SEQ ID NOs: 15-16. In some embodiments, the construct further comprises a nucleic acid sequence encoding a PPT1 protein, wherein the PPT1 protein comprises a sequence at least 85% identical to one of SEQ ID NOs: 13-14. In some embodiments, the construct further comprises a nucleic acid sequence encoding a PPT1 protein, wherein the nucleic acid sequence comprises the sequence of SEQ ID NO: 16. In some embodiments, the construct further comprises a nucleic acid sequence encoding a PPT1 protein, wherein the PPT1 protein comprises the sequence of SEQ ID NO: 14. In some embodiments, the construct further comprises a promoter sequence. In some embodiments, the promoter is a constitutive promoter. In some embodiments, the promoter is a tissue-specific promoter. In some embodiments, the construct further comprises one or more nucleic acid sequences selected from the group consisting of: a Kozak sequence, a CrPV IRES, a nucleic acid sequence encoding a linker, a nucleic acid sequence encoding a signal sequence, and a nucleic acid sequence encoding an IGF2 peptide. In some embodiments, the signal peptide sequence comprises a binding immunoglobulin protein (Bip) signal sequence. In some embodiments, the signal peptide sequence comprises the Bip signal sequence comprises an amino acid sequence at least 90% identical to an amino acid sequence selected from the group consisting of SEQ ID NOs: 29-33. In some embodiments, the construct further comprises an internal ribosomal entry sequence (IRES). In some embodiments, the IRES comprises a cricket paralysis virus (CrPV) IRES. In some embodiments, the construct further comprises a nucleic acid sequence encoding a variant IGF2 (vIGF2) peptide. In some embodiments, the vIGF2 peptide comprising an amino acid sequence at least 90% identical to an amino acid sequence selected from the group consisting of SEQ ID NOs: 17-27. In some embodiments, the nucleic acid sequence encoding the vIGF2 peptide is 5′ to the nucleic acid sequence encoding the stabilized form of the protein. In some embodiments, the nucleic acid sequence encoding the vIGF2 peptide is 3′ to the nucleic acid sequence encoding the stabilized form of the protein. In some embodiments, the construct is packaged within a viral capsid. In some embodiments, the excipient is selected from the group consisting of saline, maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate.

In further aspects, there are provided methods for treating a genetic disorder in a subject comprising administering to the subject a therapeutically effective amount of a gene therapy vector comprising a nucleic acid construct comprising: a nucleic acid encoding a stabilized form of a protein for treating a genetic disorder or a pharmaceutical compositions thereof. In some embodiments, the stabilized form comprises one or more non-native cysteine residues that form a disulfide bridge between non-native cysteines within the protein or between non-native cysteines of two monomers of the protein. In some embodiments, the protein is selected from the group consisting of alpha-galactosidase A, β-glucocerebrosidase, glucocerebrosidase, lysosomal acid lipase, glycosaminoglycan alpha-L-iduronidase, alpha-L-iduronidase, N-sulfoglucosamine sulfohydrolase (SGSH), N-acetyl-alpha-glucosaminidase (NAGLU), iduronate-2-sulfatase, N-acetylgalactosamine-6-sulfatase, glycosaminoglycan N-acetylgalactosamine 4-sulfatase, alpha-glucosidase, tripeptidyl peptidase 1 (TPP1), palmitoyl protein thioesterases (PPTs), ceroid lipofuscinoses neuronal 1, ceroid lipofuscinoses neuronal 2, ceroid lipofuscinoses neuronal 3, ceroid lipofuscinoses neuronal 4, ceroid lipofuscinoses neuronal 5, ceroid lipofuscinoses neuronal 6, ceroid lipofuscinoses neuronal 7, ceroid lipofuscinoses neuronal 8, ceroid lipofuscinoses neuronal 9, ceroid lipofuscinoses neuronal 10, ceroid lipofuscinoses neuronal 11, ceroid lipofuscinoses neuronal 12, ceroid lipofuscinoses neuronal 13, ceroid lipofuscinoses neuronal 14, ceroid lipofuscinoses neuronal 15, ceroid lipofuscinoses neuronal 16, and cyclin dependent kinase like 5. In some embodiments, the protein is selected from the group consisting of alpha-galactosidase A, β-glucocerebrosidase, glucocerebrosidase, lysosomal acid lipase, glycosaminoglycan alpha-L-iduronidase, alpha-L-iduronidase, N-sulfoglucosamine sulfohydrolase (SGSH), N-acetyl-alpha-glucosaminidase (NAGLU), iduronate-2-sulfatase, N-acetylgalactosamine-6-sulfatase, glycosaminoglycan N-acetylgalactosamine 4-sulfatase, alpha-glucosidase, tripeptidyl peptidase 1 (TPP1), palmitoyl protein thioesterases (PPTs), ceroid lipofuscinoses neuronal 4, ceroid lipofuscinoses neuronal 10 (cathepsin D), ceroid lipofuscinoses neuronal 11 (progranulin), ceroid lipofuscinoses neuronal 13 (cathepsin F), ceroid lipofuscinoses neuronal 14 (KCTD7), ceroid lipofuscinoses neuronal 15 (TBCK), and cyclin dependent kinase like 5. In some embodiments, the stabilized protein comprises a lysosomal enzyme. In some embodiments, the stabilized protein comprises a stabilized α-galactosidase (α-GAL) protein. In some embodiments, the stabilized α-galactosidase A (α-GAL) protein comprises one or more non-native cysteine residues D233C and I359C. In some embodiments, the stabilized protein comprises a stabilized palmitoyl protein thioesterase 1 (PPT1). In some embodiments, the stabilized PPT1 protein comprises non-native cysteine residues A171C and A183C. In some embodiments, the stabilized protein has a longer half-life at pH 7.4 compared to a corresponding protein without the non-native cysteines. In some embodiments, the stabilized protein can replace a protein defective or deficient in the genetic disorder. In some embodiments, the stabilized protein can reduce or slow one or more symptoms associated with the genetic disorder. In some embodiments, the stabilized protein is more effective at reducing or slowing one or more symptoms of the genetic disorder, compared to an unstabilized protein. In some embodiments, the genetic disorder is a neurological disorder. In some embodiments, the genetic disorder is a lysosomal storage disorder. In some embodiments, the genetic disorder is selected from the group consisting of aspartylglucosaminuria, Batten disease, cystinosis, Fabry disease, Gaucher disease type I, Gaucher disease type II, Gaucher disease type III, Pompe disease, Tay Sachs disease, Sandhoff disease, metachomatic leukodystrophy, mucolipidosis type I, mucolipidosis type II, mucolipidosis type III, mucolipidosis type IV, Hurler disease, Hunter disease, Sanfilippo disease type A, Sanfilippo disease type B, Sanfilippo disease type C, Sanfilippo disease type D, Morquio disease type A, Morquio disease type B, Maroteau-Lamy disease, Sly disease, Niemann-Pick disease type A, Niemann-Pick disease type B, Niemann-Pick disease type C1, Niemann-Pick disease type C2, Schindler disease type I, Schindler disease type II, adenosine deaminase severe combined immunodeficiency (ADA-SCID), chronic granulomatous disease (CGD), infantile, juvenile and adult forms of neuronal ceroid lipofuscinosis, and CDKL5 deficiency disease. In some embodiments, the gene therapy vector is a viral vector selected from the group consisting of an adenovirus vector, an adeno-associated virus vector, a retrovirus vector, a lentivirus vector, and a herpes virus vector. In some embodiments, the adeno-associated virus is a serotype selected from the group consisting of: AAV-1, AAV-2, AAV-3, AAV-4, AAV-5, AAV-6, AAV-7, AAV-8, AAV-9, AAV-10, AAV-11, AAV-12, AAV-13, AAV rh.74, AAV-B1 and AAV-hu68. In some embodiments, the nucleic acid construct is comprised in a viral vector genome. In some embodiments, the viral vector genome comprises a recombinant AAV (rAAV) genome. In some embodiments, In some embodiments, In some embodiments, In some embodiments, In some embodiments, the rAAV genome comprises a self-complementary genome. In some embodiments, the rAAV genome comprises a single-stranded genome. In some embodiments, the rAAV genome comprises a first inverted terminal repeat and a second inverted terminal repeat. In some embodiments, the AAV inverted terminal repeats are AAV2 inverted terminal repeats. In some embodiments, the rAAV genome further comprises an SV40 intron. In some embodiments, the rAAV genome further comprises a poly-adenylation sequence. In some embodiments, the construct further comprises a nucleic acid sequence encoding an α-GAL protein, wherein the nucleic acid sequence is at least 85% identical to one of SEQ ID NOs: 7-12. In some embodiments, the construct further comprises a nucleic acid sequence encoding an α-GAL protein, wherein the α-GAL protein comprises a sequence at least 85% identical to one of SEQ ID NOs: 1-6. In some embodiments, the construct further comprises a nucleic acid sequence encoding an α-GAL protein, wherein the nucleic acid sequence comprises the sequence of one of SEQ ID NOs: 8-12. In some embodiments, the construct further comprises a nucleic acid sequence encoding an α-GAL protein, wherein the α-GAL protein comprises the sequence of one of SEQ ID NOs: 2-6. In some embodiments, the construct further comprises a nucleic acid sequence encoding a PPT1 protein, wherein the nucleic acid sequence is at least 85% identical to one of SEQ ID NOs: 15-16. In some embodiments, the construct further comprises a nucleic acid sequence encoding a PPT1 protein, wherein the PPT1 protein comprises a sequence at least 85% identical to one of SEQ ID NOs: 13-14. In some embodiments, the construct further comprises a nucleic acid sequence encoding a PPT1 protein, wherein the nucleic acid sequence comprises the sequence of SEQ ID NO: 16. In some embodiments, the construct further comprises a nucleic acid sequence encoding a PPT1 protein, wherein the PPT1 protein comprises the sequence of SEQ ID NO: 14. In some embodiments, the construct further comprises a promoter sequence. In some embodiments, the promoter is a constitutive promoter. In some embodiments, the promoter is a tissue-specific promoter. In some embodiments, the construct further comprises one or more nucleic acid sequences selected from the group consisting of: a Kozak sequence, a CrPV IRES, a nucleic acid sequence encoding a linker, a nucleic acid sequence encoding a signal sequence, and a nucleic acid sequence encoding an IGF2 peptide. In some embodiments, the signal peptide sequence comprises a binding immunoglobulin protein (Bip) signal sequence. In some embodiments, the signal peptide sequence comprises the Bip signal sequence comprises an amino acid sequence at least 90% identical to an amino acid sequence selected from the group consisting of SEQ ID NOs: 29-33. In some embodiments, the construct further comprises an internal ribosomal entry sequence (IRES). In some embodiments, the IRES comprises a cricket paralysis virus (CrPV) IRES. In some embodiments, the construct further comprises a nucleic acid sequence encoding a variant IGF2 (vIGF2) peptide. In some embodiments, the vIGF2 peptide comprising an amino acid sequence at least 90% identical to an amino acid sequence selected from the group consisting of SEQ ID NOs: 17-27. In some embodiments, the nucleic acid sequence encoding the vIGF2 peptide is 5′ to the nucleic acid sequence encoding the stabilized form of the protein. In some embodiments, the nucleic acid sequence encoding the vIGF2 peptide is 3′ to the nucleic acid sequence encoding the stabilized form of the protein. In some embodiments, the construct is packaged within a viral capsid. In some embodiments, the gene therapy vector or pharmaceutical composition is delivered by intrathecal, intracerebroventricular, intraperenchymal, or intravenous injection, or a combination thereof. In some embodiments, the gene therapy vector or pharmaceutical composition reduces or slows one or more symptoms of the genetic disorder in the subject. In some embodiments, the genetic disorder is a lysosomal storage disorder. In some embodiments, the genetic disorder is selected from the group consisting of aspartylglucosaminuria, batten disease, cystinosis, Fabry disease, Gaucher disease type I, Gaucher disease type II, Gaucher disease type III, Pompe disease, Tay Sachs disease, Sandhoff disease, metachomatic leukodystrophy, mucolipidosis type I, mucolipidosis type II, mucolipidosis type III, mucolipidosis type IV, Hurler disease, Hunter disease, Sanfilippo disease type A, Sanfilippo disease type B, Sanfilippo disease type C, Sanfilippo disease type D, Morquio disease type A, Morquio disease type B, Maroteau-Lamy disease, Sly disease, Niemann-Pick disease type A, Niemann-Pick disease type B, Niemann-Pick disease type C1, Niemann-Pick disease type C2, Schindler disease type I, Schindler disease type II, adenosine deaminase severe combined immunodeficiency (ADA-SCID), chronic granulomatous disease (CGD), neuronal ceroid lipofuscinosis, and CDKL5 deficiency disorder.

In additional aspects, there are provided stabilized human α-galactosidase A (α-GAL) dimers. In some embodiments stabilized α-GAL dimers comprise one or more non-native cysteine residues, wherein the one or more non-native cysteine residues form at least one intermolecular disulfide bond connecting a first subunit and a second subunit of the α-GAL dimer. In some embodiments, the one or more non-native cysteine residues are selected from the group consisting of: (i) D233C and I359C; and (ii) M51C and G360C. In some embodiments, the one or more non-native cysteine residues comprise D233C and I359C. In some embodiments, the one or more non-native cysteine residues comprise M51C and G360C. In some embodiments, the one or more non-native cysteine residues comprise i) D233C and I359C; and (ii) M51C and G360C. In some embodiments, the polypeptide has a sequence at least 90% identical to one of SEQ ID NOs: 1-6. In some embodiments, the polypeptide is encoded by a nucleic acid at least 85% identical to one of SEQ ID NOs: 7-12. In some embodiments, the polypeptide shows increased half-life at pH 7.4 compared with a wild type α-GAL polypeptide. In some embodiments, the polypeptide further comprises a variant IGF2 (vIGF2) peptide.

In further aspects, there are provided pharmaceutical compositions comprising stabilized human α-GAL dimers and a pharmaceutically acceptable excipient, carrier, or diluent. In some embodiments stabilized α-GAL dimers comprise one or more non-native cysteine residues, wherein the one or more non-native cysteine residues form at least one intermolecular disulfide bond connecting a first subunit and a second subunit of the α-GAL dimer. In some embodiments, the one or more non-native cysteine residues are selected from the group consisting of: (i) D233C and I359C; and (ii) M51C and G360C. In some embodiments, the one or more non-native cysteine residues comprise D233C and I359C. In some embodiments, the one or more non-native cysteine residues comprise M51C and G360C. In some embodiments, the one or more non-native cysteine residues comprise i) D233C and I359C; and (ii) M51C and G360C. In some embodiments, the polypeptide has a sequence at least 90% identical to one of SEQ ID NOs: 1-6. In some embodiments, the polypeptide is encoded by a nucleic acid at least 85% identical to one of SEQ ID NOs: 7-12. In some embodiments, the polypeptide shows increased half-life at pH 7.4 compared with a wild type α-GAL polypeptide. In some embodiments, the polypeptide further comprises a variant IGF2 (vIGF2) peptide. In some embodiments, the excipient is selected from the group consisting of saline, maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate.

In additional aspects, there are provided methods for treating Fabry disease in a subject comprising administering to the subject a therapeutically effective amount of a stabilized human α-GAL dimer or pharmaceutical composition thereof to a subject in need thereof. In some embodiments stabilized α-GAL dimers comprise one or more non-native cysteine residues, wherein the one or more non-native cysteine residues form at least one intermolecular disulfide bond connecting a first subunit and a second subunit of the α-GAL dimer. In some embodiments, the one or more non-native cysteine residues are selected from the group consisting of: (i) D233C and I359C; and (ii) M51C and G360C. In some embodiments, the one or more non-native cysteine residues comprise D233C and I359C. In some embodiments, the one or more non-native cysteine residues comprise M51C and G360C. In some embodiments, the one or more non-native cysteine residues comprise i) D233C and I359C; and (ii) M51C and G360C. In some embodiments, the polypeptide has a sequence at least 90% identical to one of SEQ ID NOs: 1-6. In some embodiments, the polypeptide is encoded by a nucleic acid at least 85% identical to one of SEQ ID NOs: 7-12. In some embodiments, the polypeptide shows increased half-life at pH 7.4 compared with a wild type α-GAL polypeptide. In some embodiments, the polypeptide further comprises a variant IGF2 (vIGF2) peptide. In some embodiments, the excipient is selected from the group consisting of saline, maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate. In some embodiments, the stabilized human α-GAL dimer or pharmaceutical composition is delivered by intrathecal, intracerebroventricular, intraperenchymal, subcutaneous, intramuscular, ocular, intravenous injection, or a combination thereof. In some embodiments, the stabilized human α-GAL dimer or pharmaceutical composition reduces or slows one or more symptoms of the Fabry disease in the subject.

In additional aspects, there are provided stabilized human palmitoyl protein thioesterase 1 (PPT1) molecules. In some embodiments, the stabilized PPT1 molecule comprises one or more non-native cysteine residues wherein the one or more non-native cysteine residues form at least one intramolecular disulfide bond within the PPT1 molecule. In some embodiments, the stabilized PPT1 comprises non-native cysteine residues A171C and A183C. In some embodiments, the polypeptide has a sequence at least 90% identical to one of SEQ ID NOs: 13-14. In some embodiments, the polypeptide is encoded by a nucleic acid at least 85% identical to one of SEQ ID NOs: 15-16. In some embodiments, the polypeptide shows increased half-life at pH 7.4 compared with a wild type PPT1 polypeptide. In some embodiments, the polypeptide further comprises a variant IGF2 (vIGF2) peptide.

In further aspects, there are provided pharmaceutical compositions comprising a stabilized PPT1 and a pharmaceutically acceptable excipient, carrier, or diluent. In some embodiments, the stabilized PPT1 molecule comprises one or more non-native cysteine residues wherein the one or more non-native cysteine residues form at least one intramolecular disulfide bond within the PPT1 molecule. In some embodiments, the stabilized PPT1 comprises non-native cysteine residues A171C and A183C. In some embodiments, the polypeptide has a sequence at least 90% identical to one of SEQ ID NOs: 13-14. In some embodiments, the polypeptide is encoded by a nucleic acid at least 85% identical to one of SEQ ID NOs: 15-16. In some embodiments, the polypeptide shows increased half-life at pH 7.4 compared with a wild type PPT1 polypeptide. In some embodiments, the polypeptide further comprises a variant IGF2 (vIGF2) peptide. In some embodiments, the excipient is selected from the group consisting of saline, maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate.

In additional aspects, there are provided methods for treating CLN1 disease in a subject comprising administering to the subject a therapeutically effective amount of a stabilized PPT1 or a pharmaceutical composition thereof to a subject in need thereof. In some embodiments, the stabilized PPT1 molecule comprises one or more non-native cysteine residues wherein the one or more non-native cysteine residues form at least one intramolecular disulfide bond within the PPT1 molecule. In some embodiments, the stabilized PPT1 comprises non-native cysteine residues A171C and A183C. In some embodiments, the polypeptide has a sequence at least 90% identical to one of SEQ ID NOs: 13-14. In some embodiments, the polypeptide is encoded by a nucleic acid at least 85% identical to one of SEQ ID NOs: 15-16. In some embodiments, the polypeptide shows increased half-life at pH 7.4 compared with a wild type PPT1 polypeptide. In some embodiments, the polypeptide further comprises a variant IGF2 (vIGF2) peptide. In some embodiments, the excipient is selected from the group consisting of saline, maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate. In some embodiments, the modified PPT1 or pharmaceutical composition is delivered by intrathecal, intracerebroventricular, intraperenchymal, subcutaneous, intramuscular, ocular, intravenous injection, or a combination thereof.

In additional aspects, there are provided modified human α-galactosidase A (α-GAL) polypeptides comprising cysteine substitutions of an α-GAL polypeptide sequence selected from the group consisting of: (i) D233C and I359C; and (ii) M51C and G360C. In some embodiments, the polypeptide comprises cysteine substitutions of an α-GAL polypeptide sequence of D233C and I359C. In some embodiments, the polypeptide comprises cysteine substitutions of an α-GAL polypeptide sequence of M51C and G360C. In some embodiments, the polypeptide forms a homodimer. In some embodiments, the homodimer is stabilized by a disulfide bond. In some embodiments, the polypeptide shows increased half-life at pH 7.4 compared with a wild type α-GAL polypeptide.

In further aspects, there are provided, nucleic acid molecules comprising a nucleic acid encoding a modified human α-GAL polypeptide. In some embodiments, the modified human α-GAL polypeptide comprises cysteine substitutions of an α-GAL polypeptide sequence selected from the group consisting of: (i) D233C and I359C; and (ii) M51C and G360C. In some embodiments, the polypeptide comprises cysteine substitutions of an α-GAL polypeptide sequence of D233C and I359C. In some embodiments, the polypeptide comprises cysteine substitutions of an α-GAL polypeptide sequence of M51C and G360C. In some embodiments, the polypeptide forms a homodimer. In some embodiments, the homodimer is stabilized by a disulfide bond. In some embodiments, the polypeptide shows increased half-life at pH 7.4 compared with a wild type α-GAL polypeptide.

In further aspects, there are provided gene therapy vectors comprising a nucleic acid molecule comprising a nucleic acid encoding a modified human α-GAL polypeptide. In some embodiments, the modified human α-GAL polypeptide comprises cysteine substitutions of an α-GAL polypeptide sequence selected from the group consisting of: (i) D233C and I359C; and (ii) M51C and G360C. In some embodiments, the polypeptide comprises cysteine substitutions of an α-GAL polypeptide sequence of D233C and I359C. In some embodiments, the polypeptide comprises cysteine substitutions of an α-GAL polypeptide sequence of M51C and G360C. In some embodiments, the polypeptide forms a homodimer. In some embodiments, the homodimer is stabilized by a disulfide bond. In some embodiments, the polypeptide shows increased half-life at pH 7.4 compared with a wild type α-GAL polypeptide.

In additional aspects, there are provided, modified human α-galactosidase A (α-GAL) polypeptides comprising cysteine substitutions of an α-GAL polypeptide sequence, wherein the cysteine substitutions facilitate disulfide bond formation between two α-GAL polypeptides to form a homodimer. In some embodiments, the polypeptide comprises cysteine substitutions of an α-GAL polypeptide sequence selected from the group consisting of: (i) D233C and I359C; and (ii) M51C and G360C. In some embodiments, the polypeptide comprises cysteine substitutions of an α-GAL polypeptide sequence of D233C and 1359C. In some embodiments, the polypeptide comprises cysteine substitutions of an α-GAL polypeptide sequence of M51C and G360C. In some embodiments, the polypeptide shows increased half-life at pH 7.4 compared with a wild type α-GAL polypeptide.

In further aspects, there are provided nucleic acid molecule comprising a nucleic acid encoding a modified human α-GAL polypeptide. In some embodiments, the modified human α-galactosidase A (α-GAL) polypeptides comprise cysteine substitutions of an α-GAL polypeptide sequence, wherein the cysteine substitutions facilitate disulfide bond formation between two α-GAL polypeptides to form a homodimer. In some embodiments, the polypeptide comprises cysteine substitutions of an α-GAL polypeptide sequence selected from the group consisting of: (i) D233C and I359C; and (ii) M51C and G360C. In some embodiments, the polypeptide comprises cysteine substitutions of an α-GAL polypeptide sequence of D233C and I359C. In some embodiments, the polypeptide comprises cysteine substitutions of an α-GAL polypeptide sequence of M51C and G360C. In some embodiments, the polypeptide shows increased half-life at pH 7.4 compared with a wild type α-GAL polypeptide.

In additional aspects, there are provided gene therapy vectors comprising a nucleic acid encoding a modified human α-GAL polypeptide. In some embodiments, the modified human α-galactosidase A (α-GAL) polypeptide comprises cysteine substitutions of an α-GAL polypeptide sequence, wherein the cysteine substitutions facilitate disulfide bond formation between two α-GAL polypeptides to form a homodimer. In some embodiments, the polypeptide comprises cysteine substitutions of an α-GAL polypeptide sequence selected from the group consisting of: (i) D233C and I359C; and (ii) M51C and G360C. In some embodiments, the polypeptide comprises cysteine substitutions of an α-GAL polypeptide sequence of D233C and I359C. In some embodiments, the polypeptide comprises cysteine substitutions of an α-GAL polypeptide sequence of M51C and G360C. In some embodiments, the polypeptide shows increased half-life at pH 7.4 compared with a wild type α-GAL polypeptide.

In further aspects, there are provided homodimers comprising two modified human α-GAL polypeptides, wherein each modified human α-GAL polypeptide comprises cysteine substitutions of an α-GAL polypeptide sequence selected from the group consisting of: (i) D233C and I359C; and (ii) M51C and G360C. In some embodiments, each modified human α-GAL polypeptide comprises cysteine substitutions of an α-GAL polypeptide sequence of D233C and I359C. In some embodiments, each modified human α-GAL polypeptide comprises cysteine substitutions of an α-GAL polypeptide sequence of M51C and G360C. In some embodiments, the homodimer is stabilized by a disulfide bond. In some embodiments, the homodimer shows increased half-life at pH 7.4 compared with a wild type α-GAL homodimer.

In additional aspects, there are provided homodimers comprising two modified human α-GAL polypeptides, wherein each modified human α-GAL polypeptide comprises cysteine substitutions of an α-GAL polypeptide sequence, wherein the cysteine substitutions facilitate disulfide bond formation between two α-GAL polypeptides to form a homodimer. In some embodiments, the polypeptide comprises cysteine substitutions of an α-GAL polypeptide sequence selected from the group consisting of: (i) D233C and I359C; and (ii) M51C and G360C. In some embodiments, each modified human α-GAL polypeptide comprises cysteine substitutions of an α-GAL polypeptide sequence of D233C and I359C. In some embodiments, each modified human α-GAL polypeptide comprises cysteine substitutions of an α-GAL polypeptide sequence of M51C and G360C. In some embodiments, the homodimer is stabilized by a disulfide bond. In some embodiments, the homodimer shows increased half-life at pH 7.4 compared with a wild type α-GAL homodimer.

In further aspects, there are provided nucleic acid molecules comprising a nucleic acid encoding a modified human α-GAL polypeptide, wherein the nucleic acid encodes a polypeptide comprising cysteine substitutions of an α-GAL polypeptide sequence selected from the group consisting of: (i) D233C and I359C; and (ii) M51C and G360C. In some embodiments, the nucleic acid encodes a polypeptide comprising cysteine substitutions of D233C and I359C. In some embodiments, the nucleic acid encodes a polypeptide comprising cysteine substitutions of M51C and G360C. In some embodiments, the polypeptide forms a homodimer. In some embodiments, the homodimer is stabilized by a disulfide bond. In some embodiments, the polypeptide shows increased half-life at pH 7.4 compared with a wild type α-GAL polypeptide. In some embodiments, the nucleic acid is a gene therapy construct. In some embodiments, the nucleic acid further comprises a promoter. In some embodiments, the promoter is a constitutive promoter. In some embodiments, the promoter is a tissue-specific promoter. In some embodiments, the nucleic acid comprises at least a portion of a virus nucleic acid sequence. In some embodiments, the virus is selected from wherein the virus comprises a retrovirus, an adenovirus, an adeno associated virus, a lentivirus, or a herpes virus.

In additional aspects, there are provided nucleic acid molecules comprising a nucleic acid encoding a modified human α-GAL polypeptide, wherein the nucleic acid encodes a polypeptide comprising cysteine substitutions of an α-GAL polypeptide sequence, wherein the cysteine substitutions facilitate disulfide bond formation between two α-GAL polypeptides to form a homodimer. In some embodiments, the polypeptide comprises cysteine substitutions of an α-GAL polypeptide sequence selected from the group consisting of: (i) D233C and I359C; and (ii) M51C and G360C. In some embodiments, the nucleic acid encodes a polypeptide comprising cysteine substitutions of D233C and I359C. In some embodiments, the nucleic acid encodes a polypeptide comprising cysteine substitutions of M51C and G360C. In some embodiments, the polypeptide forms a homodimer. In some embodiments, the homodimer is stabilized by a disulfide bond. In some embodiments, the polypeptide shows increased half-life at pH 7.4 compared with a wild type α-GAL polypeptide. In some embodiments, the nucleic acid is a gene therapy construct. In some embodiments, the nucleic acid further comprises a promoter. In some embodiments, the promoter is a constitutive promoter. In some embodiments, the promoter is a tissue-specific promoter. In some embodiments, the nucleic acid comprises at least a portion of a virus nucleic acid sequence. In some embodiments, the virus is selected from wherein the virus comprises a retrovirus, an adenovirus, an adeno associated virus, a lentivirus, or a herpes virus.

In additional aspects, there are provided nucleic acid constructs comprising at least one promoter and a nucleic acid encoding a modified human α-GAL polypeptide, wherein the modified human α-GAL polypeptide comprises cysteine substitutions of an α-GAL polypeptide sequence selected from the group consisting of: (i) D233C and I359C; and (ii) M51C and G360C. In some embodiments, the nucleic acid encodes a polypeptide comprising cysteine substitutions of an α-GAL polypeptide sequence of D233C and I359C. In some embodiments, the nucleic acid encodes a polypeptide comprising cysteine substitutions of an α-GAL polypeptide sequence of M51C and G360C. In some embodiments, the promoter is a constitutive promoter. In some embodiments, the promoter is a tissue-specific promoter. In some embodiments, the nucleic acid construct comprises one or more nucleic acids from the group consisting of: a CrPV IRES, a kozak sequence, a nucleic acid encoding a linker, a nucleic acid sequence encoding a leader sequence, and a nucleic acid encoding a IGF2 peptide. In some embodiments, the nucleic acid construct comprises at least a portion of a virus nucleic acid sequence. In some embodiments, the virus comprises a retrovirus, an adenovirus, an adeno associated virus, a lentivirus, or a herpes virus. In some embodiments, the polypeptide forms a homodimer. In some embodiments, the homodimer is stabilized by a disulfide bond. In some embodiments, the polypeptide shows increased half-life at pH 7.4 compared with a wild type α-GAL polypeptide. In some embodiments, the nucleic acid is packaged within in a viral capsid protein. In some embodiments, the nucleic acid construct is suitable for gene therapy.

In further aspects, there are provided nucleic acid constructs comprising at least one promoter and a nucleic acid encoding a modified human α-GAL polypeptide, wherein the modified human α-GAL polypeptide comprises cysteine substitutions of an α-GAL polypeptide sequence, wherein the cysteine substitutions facilitate disulfide bond formation between two α-GAL polypeptides to form a homodimer. In some embodiments, the polypeptide comprises cysteine substitutions of an α-GAL polypeptide sequence selected from the group consisting of: (i) D233C and I359C; and (ii) M51C and G360C. In some embodiments, the nucleic acid encodes a polypeptide comprising cysteine substitutions of an α-GAL polypeptide sequence of D233C and I359C. In some embodiments, the nucleic acid encodes a polypeptide comprising cysteine substitutions of an α-GAL polypeptide sequence of M51C and G360C. In some embodiments, the promoter is a constitutive promoter. In some embodiments, the promoter is a tissue-specific promoter. In some embodiments, the nucleic acid construct comprises one or more nucleic acids from the group consisting of: a CrPV IRES, a kozak sequence, a nucleic acid encoding a linker, a nucleic acid sequence encoding a leader sequence, and a nucleic acid encoding a IGF2 peptide. In some embodiments, the nucleic acid construct comprises at least a portion of a virus nucleic acid sequence. In some embodiments, the virus comprises a retrovirus, an adenovirus, an adeno associated virus, a lentivirus, or a herpes virus. In some embodiments, the polypeptide forms a homodimer. In some embodiments, the homodimer is stabilized by a disulfide bond. In some embodiments, the polypeptide shows increased half-life at pH 7.4 compared with a wild type α-GAL polypeptide. In some embodiments, the nucleic acid is packaged within in a viral capsid protein. In some embodiments, the nucleic acid construct is suitable for gene therapy.

In further aspects, there are provided pharmaceutical compositions comprising (a) a modified human α-GAL polypeptide, wherein the modified human α-GAL polypeptide comprises cysteine substitutions of an α-GAL polypeptide sequence selected from the group consisting of: (i) D233C and I359C; and (ii) M51C and G360C and (b) a pharmaceutically acceptable excipient. In some embodiments, the modified human α-GAL polypeptide comprises cysteine substitutions of an α-GAL polypeptide sequence of D233C and 1359C. In some embodiments, the modified human α-GAL polypeptide comprises cysteine substitutions of an α-GAL polypeptide sequence of M51C and G360C. In some embodiments, the modified human α-GAL polypeptide forms a homodimer. In some embodiments, the homodimer is stabilized by a disulfide bond. In some embodiments, the modified human α-GAL polypeptide shows increased half-life at pH 7.4 compared with a wild type α-GAL polypeptide. In some embodiments, the excipient is selected from the group consisting of saline, maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate. In some embodiments, the composition is suitable for enzyme replacement therapy.

In additional aspects, there are provided methods of ameliorating at least one symptom of Fabry disease in a subject in need thereof, the method comprising administering at least one dose of a composition comprising a gene therapy nucleic acid construct comprising at least one promoter and a nucleic acid encoding a modified human α-GAL polypeptide, wherein the modified human α-GAL polypeptide comprises cysteine substitutions of an α-GAL polypeptide sequence selected from the group consisting of: (i) D233C and I359C; and (ii) M51C and G360C. In some embodiments, the nucleic acid encodes a polypeptide comprising cysteine substitutions of an α-GAL polypeptide sequence of D233C and I359C. In some embodiments, the nucleic acid encodes a polypeptide comprising cysteine substitutions of an α-GAL polypeptide sequence of M51C and G360C. In some embodiments, the nucleic acid encodes a polypeptide which forms a homodimer. In some embodiments, the homodimer is stabilized by a disulfide bond. In some embodiments, the nucleic acid encodes a modified human α-GAL polypeptide having increased half-life at pH 7.4 compared with a wild type α-GAL polypeptide. In some embodiments, the promoter is a constitutive promoter. In some embodiments, the promoter is a tissue-specific promoter. In some embodiments, the nucleic acid comprises at least a portion of a virus. In some embodiments, the virus is selected from wherein the virus comprises a retrovirus, an adenovirus, an adeno associated virus, a lentivirus, or a herpes virus. In some embodiments, the nucleic acid is packaged within in a viral capsid protein. In some embodiments, the at least one symptom is selected from one or more of pain, skin discoloration, inability to sweat, eye cloudiness, gastrointestinal dysfunction, tinnitus, hearing loss, mitral valve prolapse, heart disease, joint pain, renal failure, and kidney dysfunction. In some embodiments, at least one symptom is reduced with a single administration of the gene therapy nucleic acid construct. In some embodiments, the method further comprises measuring an α-GAL activity in a tissue obtained from the subject following treatment.

In further aspects, there are provided methods of ameliorating at least one symptom of Fabry disease in a subject in need thereof, the method comprising administering at least one dose of a composition comprising a modified human α-GAL polypeptide, wherein the modified human α-GAL polypeptide comprises cysteine substitutions of an α-GAL polypeptide sequence selected from the group consisting of: (i) D233C and I359C; and (ii) M51C and G360C. In some embodiments, the modified human α-GAL polypeptide cysteine substitutions of an α-GAL polypeptide sequence of D233C and I359C. In some embodiments, the modified human α-GAL polypeptide cysteine substitutions of an α-GAL polypeptide sequence of M51C and G360C. In some embodiments, the modified human α-GAL polypeptide forms a homodimer. In some embodiments, the homodimer is stabilized by a disulfide bond. In some embodiments, the modified human α-GAL polypeptide shows increased half-life at pH 7.4 compared with a wild type α-GAL polypeptide. In some embodiments, the at least one symptom is selected from one or more of pain, skin discoloration, inability to sweat, eye cloudiness, gastrointestinal dysfunction, tinnitus, hearing loss, mitral valve prolapse, heart disease, joint pain, renal failure, and kidney dysfunction.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1A and FIG. 1B show the structure of α-galactosidase A (α-GAL) and the proposed sites of amino acid substitutions.

FIG. 2A shows modified α-GAL dimer formation.

FIG. 2B shows modified α-GAL enzymatic activity.

FIG. 3A shows stability of α-GAL at pH 4.6 and pH 7.4 over 24 hours.

FIG. 3B shows stability of α-GAL at pH 4.6 and pH 7.4 over 7 days.

FIG. 4A shows uptake and enzymatic activity of modified α-GAL of Fabry patient fibroblasts.

FIG. 4B shows reduction of globotriaosylsphingosine (lyso-Gb₃) in Fabry patient fibroblasts.

FIG. 5 shows activity of enhanced half-life of two α-GAL disulfide dimers in Fabry disease cells.

FIG. 6 shows GB3 substrate histology in wildtype mice and GLA knockout mice with and without treatment with modified α-GAL gene therapy.

FIG. 7 shows GLA enzyme activity in wildtype mice and GLA knockout mice with and without treatment with modified α-GAL gene therapy.

FIG. 8 shows GB3 substrate measured in kidney tissue lysate in wildtype mice and GLA knockout mice with and without treatment with modified α-GAL gene therapy.

FIG. 9 shows WinNonlin analysis of enzymatic activity of palmitoyl protein thioesterase 1 (PPT-1) wildtype vs Construct PPT-1 mutant over time.

### DETAILED DESCRIPTION

Provided herein are variants of polypeptides for therapeutics including constructs for gene therapy having cysteine substitutions which enable stabilization due to formation of disulfide bonds within the molecule or to disulfide bonds forming between the two subunits in the polypeptide to form a dimer. These disulfide bonds result in a more stable recombinant enzyme at neutral pH, such as the pH of blood. Accordingly, a more stable polypeptide with longer half-life is provided that is useful for treatment of diseases resulting from mutations, including diseases resulting from mutation of α-GAL, such as Fabry disease; or mutation of PPT-1, such as CLN1 disease. Polypeptide variants (also termed “modified polypeptides”) herein include but are not limited to variants of α-GAL and PPT-1.

### Modified α-GAL Polypeptides

Provided herein are modified α-GAL polypeptides comprising cysteine substitutions of an α-GAL polypeptide sequence. Contemplated substitutions provided herein include: (i) R49C and G361C; (ii) R49C and G360C; (iii) D233C and I359C; (iv) M51C and G360C; and (v) S276C. In some embodiments, the polypeptide comprises cysteine substitutions of an α-GAL polypeptide sequence selected from the group consisting of: (i) D233C and I359C; and (ii) M51C and G360C. In some embodiments, the polypeptide comprises cysteine substitutions of an α-GAL polypeptide sequence of D233C and I359C. In some embodiments, the polypeptide comprises cysteine substitutions of an α-GAL polypeptide sequence of M51C and G360C. The modified α-GAL polypeptides a can form a homodimer is stabilized by at least one, more preferably two intermolecular disulfide bonds. The modified α-GAL polypeptides polypeptide shows increased half-life at pH 7.4 compared with a wildtype α-GAL polypeptide.

Wild type and exemplary Modified α-GAL sequences are provided in Table 1

TABLE 1
α-GAL Polypeptide Sequences

| | | SEQ |
|---|---|---|
| α-GAL | | ID |
| variant | Sequence | NO: |
| Human | MQLRNPELHLGCALALRFLALVSWDIPGARALD | 1 |
| α-GAL | NGLARTPTMGWLHWERFMCNLDCQEEPDSCISE | |
| wild | KLFMEMAELMVSEGWKDAGYEYLCIDDCWMAPQ | |
| type | RDSEGRLQADPQRFPHGIRQLANYVHSKGLKLG | |
| (NP_ | IYADVGNKTCAGFPGSFGYYDIDAQTFADWGVD | |
| 000160.1) | LLKFDGCYCDSLENLADGYKHMSLALNRTGRSI | |
| | VYSCEWPLYMWPFQKPNYTEIRQYCNHWRNFAD | |
| | IDDSWKSIKSILDWTSFNQERIVDVAGPGGWND | |
| | PDMLVIGNFGLSWNQQVTQMALWAIMAAPLFMS | |
| | NDLRHISPQAKALLQDKDVIAINQDPLGKQGYQ | |
| | LRQGDNFEVWERPLSGLAWAVAMINRQEIGGPR | |
| | SYTIAVASLGKGVACNPACFITQLLPVKRKLGF | |
| | YEWTSRLRSHINPTGTVLLQLENTMQMSLKDLL | |
| Human | MQLRNPELHLGCALALRFLALVSWDIPGARALD | 2 |
| α-GAL | NGLARTPTMGWLHWECFMCNLDCQEEPDSCISE | |
| R49C- | KLFMEMAELMVSEGWKDAGYEYLCIDDCWMAPQ | |
| G361C | RDSEGRLQADPQRFPHGIRQLANYVHSKGLKLG | |
| | YADVGNKTCAGFPGSFGYYDIDAQTFAIDWGVD | |
| | LLKFDGCYCDSLENLADGYKHMSLALNRTGRSI | |
| | VYSCEWPLYMWPFQKPNYTEIRQYCNHWRNFAD | |
| | IDDSWKSIKSILDWTSFNQERIVDVAGPGGWND | |
| | PDMLVIGNFGLSWNQQVTQMALWAIMAAPLFMS | |
| | NDLRHISPQAKALLQDKDVIAINQDPLGKQGYQ | |
| | LRQGDNFEVWERPLSGLAWAVAMINRQEIGCPR | |
| | SYTIAVASLGKGVACNPACFITQLLPVKRKLGF | |
| | YEWTSRLRSHINPTGTVLLQLENTMQMSLKDLL | |
| Human | MQLRNPELHLGCALALRFLALVSWDIPGARALD | 3 |
| A-GAL | NGLARTPTMGWLHWECFMCNLDCQEEPDSCISE | |
| R49C- | KLFMEMAELMVSEGWKDAGYEYLCIDDCWMAPQ | |
| G360C | RDSEGRLQADPQRFPHGIRQLANYVHSKGLKLG | |
| | IYADVGNKTCAGFPGSFGYYDIDAQTFADWGVD | |
| | LLKFDGCYCDSLENLADGYKHMSLALNRTGRSI | |
| | VYSCEWPLYMWPFQKPNYTEIRQYCNHWRNFAD | |
| | IDDSWKSIKSILDWTSFNQERIVDVAGPGGWND | |
| | PDMLVIGNFGLSWNQQVTQMALWAIMAAPLFMS | |
| | NDLRHISPQAKALLQDKDVIAINQDPLGKQGYQ | |
| | LRQGDNFEVWERPLSGLAWAVAMINRQEICGPR | |
| | SYTIAVASLGKGVACNPACFITQLLPVKRKLGF | |
| | YEWTSRLRSHINPTGTVLLQLENTMQMSLKDLL | |
| Human | MQLRNPELHLGCALALRFLALVSWDIPGARALD | 4 |
| α-GAL | NGLARTPTMGWLHWERFCCNLDCQEEPDSCISE | |
| M51C- | KLFMEMAELMVSEGWKDAGYEYLCIDDCWMAPQ | |
| G360C | RDSEGRLQADPQRFPHGIRQLANYVHSKGLKLG | |
| | IYADVGNKTCAGFPGSFGYYDIDAQTFADWGVD | |
| | LLKFDGCYCDSLENLADGYKHMSLALNRTGRSI | |
| | VYSCEWPLYMWPFQKPNYTEIRQYCNHWRNFAD | |
| | IDDSWKSIKSILDWTSFNQERIVDVAGPGGWND | |
| | PDMLVIGNFGLSWNQQVTQMALWAIMAAPLFMS | |
| | NDLRHISPQAKALLQDKDVIAINQDPLGKQGYQ | |
| | LRQGDNFEVWERPLSGLAWAVAMINRQEICGPR | |
| | SYTIAVASLGKGVACNPACFITQLLPVKRKLGF | |
| | YEWTSRLRSHINPTGTVLLQLENTMQMSLKDLL | |
| Human | MQLRNPELHLGCALALRFLALVSWDIPGARALD | 5 |
| α-GAL | NGLARTPTMGWLHWERFMCNLDCQEEPDSCISE | |
| D233C- | KLFMEMAELMVSEGWKDAGYEYLCIDDCWMAPQ | |
| I359C | RDSEGRLQADPQRFPHGIRQLANYVHSKGLKLG | |
| | IYADVGNKTCAGFPGSFGYYDIDAQTFADWGVD | |
| | LLKFDGCYCDSLENLADGYKHMSLALNRTGRSI | |
| | VYSCEWPLYMWPFQKPNYTEIRQYCNHWRNFAD | |
| | ICDSWKSIKSILDWTSFNQERIVDVAGPGGWND | |
| | PDMLVIGNFGLSWNQQVTQMALWAIMAAPLFMS | |
| | NDLRHISPQAKALLQDKDVIAINQDPLGKQGYQ | |
| | LRQGDNFEVWERPLSGLAWAVAMINRQECGGPR | |
| | SYTIAVASLGKGVACNPACFITQLLPVKRKLGF | |
| | YEWTSRLRSHINPTGTVLLQLENTMQMSLKDLL | |
| Human | MQLRNPELHLGCALALRFLALVSWDIPGARALD | 6 |
| α-GAL | NGLARTPTMGWLHWERFMCNLDCQEEPDSCISE | |
| S276C | KLFMEMAELMVSEGWKDAGYEYLCIDDCWMAPQ | |
| | RDSEGRLQADPQRFPHGIRQLANYVHSKGLKLG | |
| | IYADVGNKTCAGFPGSFGYYDIDAQTFADWGVD | |
| | LLKFDGCYCDSLENLADGYKHMSLALNRTGRSI | |
| | VYSCEWPLYMWPFQKPNYTEIRQYCNHWRNFAD | |
| | IDDSWKSIKSILDWTSFNQERIVDVAGPGGWND | |
| | PDMLVIGNFGLCWNQQVTQMALWAIMAAPLFMS | |
| | NDLRHISPQAKALLQDKDVIAINQDPLGKQGYQ | |
| | LRQGDNFEVWERPLSGLAWAVAMINRQEIGGPR | |
| | SYTIAVASLGKGVACNPACFITQLLPVKRKLGF | |
| | YEWTSRLRSHINPTGTVLLQLENTMQMSLKDLL | |

Also provided herein are modified α-GAL polypeptides comprising a polypeptide with a sequence containing cysteine residues at positions 51 and 360 and having at least 90% identity to a sequence set forth as SEQ ID NO: 4. In some embodiments, modified α-GAL polypeptides comprise a polypeptide with a sequence containing cysteine residues at positions 51 and 360 and having at least 95% identity to a sequence set forth as SEQ ID NO: 4. In some embodiments, modified α-GAL polypeptides comprise a polypeptide with a sequence containing cysteine residues at positions 51 and 360 and having at least 96% identity to a sequence set forth as SEQ ID NO: 4. In some embodiments, modified α-GAL polypeptides comprise a polypeptide with a sequence containing cysteine residues at positions 51 and 360 and having at least 97% identity to a sequence set forth as SEQ ID NO: 4. In some embodiments, modified α-GAL polypeptides comprise a polypeptide with a sequence containing cysteine residues at positions 51 and 360 and having at least 98% identity to a sequence set forth as SEQ ID NO: 4. In some embodiments, modified α-GAL polypeptides comprise a polypeptide with a sequence containing cysteine residues at positions 51 and 360 and having at least 99% identity to a sequence set forth as SEQ ID NO: 4. In some embodiments, modified α-GAL polypeptides comprise a polypeptide with a sequence containing cysteine residues at positions 51 and 360 and having more than 99% identity to a sequence set forth as SEQ ID NO: 4. In some embodiments, modified α-GAL polypeptides comprise a polypeptide with a sequence set forth as SEQ ID NO: 4.

Also provided herein are modified α-GAL polypeptides comprising a polypeptide with a sequence containing cysteine residues at positions 233 and 359 and having at least 90% identity to a sequence set forth as SEQ ID NO: 5. In some embodiments, modified α-GAL polypeptides comprise a polypeptide with a sequence containing cysteine residues at positions 233 and 359 and having at least 95% identity to a sequence set forth as SEQ ID NO: 5. In some embodiments, modified α-GAL polypeptides comprise a polypeptide with a sequence containing cysteine residues at positions 233 and 359 and having at least 96% identity to a sequence set forth as SEQ ID NO: 5. In some embodiments, modified α-GAL polypeptides comprise a polypeptide with a sequence containing cysteine residues at positions 233 and 359 and having at least 97% identity to a sequence set forth as SEQ ID NO: 5. In some embodiments, modified α-GAL polypeptides comprise a polypeptide with a sequence containing cysteine residues at positions 233 and 359 and having at least 98% identity to a sequence set forth as SEQ ID NO: 5. In some embodiments, modified α-GAL polypeptides comprise a polypeptide with a sequence containing cysteine residues at positions 233 and 359 and having at least 99% identity to a sequence set forth as SEQ ID NO: 5. In some embodiments, modified α-GAL polypeptides comprise a polypeptide with a sequence containing cysteine residues at positions 233 and 359 and having more than 99% identity to a sequence set forth as SEQ ID NO: 5. In some embodiments, modified α-GAL polypeptides comprise a polypeptide with a sequence set forth as SEQ ID NO: 5.

Also provided herein are homodimers comprising two modified α-GAL polypeptides, wherein each modified α-GAL polypeptide comprises cysteine substitutions of an α-GAL polypeptide sequence selected from the group consisting of: (i) R49C and G361C; (ii) R49C and G360C; (iii) D233C and I359C; (iv) M51C and G360C; and (v) S276C. In some embodiments, each modified α-GAL polypeptide comprises cysteine substitutions of an α-GAL polypeptide sequence selected from the group consisting of: (i) D233C and I359C; and (ii) M51C and G360C. In some embodiments, each modified α-GAL polypeptide comprises cysteine substitutions of an α-GAL polypeptide sequence of D233C and I359C. In some embodiments, each modified α-GAL polypeptide comprises cysteine substitutions of an α-GAL polypeptide sequence of M51C and G360C. In some embodiments, the homodimer is stabilized by a disulfide bond. In some embodiments, the homodimer shows increased half-life at pH 7.4 compared with a wild type α-GAL homodimer.

In some embodiments, modified α-GAL polypeptides have an increased half-life at pH 4.6. In some embodiments, the half-life at pH 4.6 is at least 50% greater than a wild type α-GAL polypeptide. In some embodiments, the half-life at pH 4.6 is at least 150% greater than a wild type α-GAL polypeptide. In some embodiments, the half-life at pH 4.6 is at least 200% greater than a wild type α-GAL polypeptide. In some embodiments, the half-life at pH 4.6 is at least 250% greater than a wild type α-GAL polypeptide. In some embodiments, the half-life at pH 4.6 is at least 300% greater than a wild type α-GAL polypeptide. In some embodiments, the half-life at pH 4.6 is at least 350% greater than a wild type α-GAL polypeptide. In some embodiments, the half-life at pH 4.6 is at least 400% greater than a wild type α-GAL polypeptide.

In some embodiments, the modified α-GAL dimer has a half-life at pH 4.6 that is increased by at least a factor of about 2, 2.5, 3, 3.5, 4, 4.5 or 5 compared to the half-life of wild type α-GAL at pH 4.6. More preferably, the modified α-GAL dimer has a half-life at pH 4.6 that is increased by at least a factor of about 3, 3.5, or 4 compared to the half-life of wild type α-GAL polypeptide at pH 4.6.

In some embodiments, the modified α-GAL dimer has an intracellular half-life at that is increased by at least a factor of about 2, 2.5, 3, 3.5, 4, 4.5 or 5 compared to the intracellular half-life of wild type human α-GAL. More preferably, the modified α-GAL dimer has an intracellular half-life that is increased by at least a factor of about 3, 3.5, or 4, 4.5 or 5 compared to the intracellular half-life of wild type α-GAL polypeptide.

The modified α-GAL dimer has a substantially increased half-life at pH 7.4 compared to wild type human α-GAL.

### Nucleic Acids Encoding Modified α-GAL Polypeptides

Also provided herein are nucleic acid molecules comprising nucleic acids encoding a modified α-GAL polypeptide. Contemplated nucleic acids include those encoding a polypeptide comprising cysteine substitutions of an α-GAL polypeptide sequence including: (i) R49C and G361C; (ii) R49C and G360C; (iii) D233C and I359C; (iv) M51C and G360C; and (v) S276C. In some embodiments, the nucleic acid encodes a polypeptide comprising cysteine substitutions of an α-GAL polypeptide sequence selected from the group consisting of: (i) D233C and I359C; and (ii) M51C and G360C. In some embodiments, the nucleic acid encodes a polypeptide comprising cysteine substitutions of D233C and I359C. In some embodiments, the nucleic acid encodes a polypeptide comprising cysteine substitutions of M51C and G360C. In some embodiments, the polypeptide forms a homodimer. In some embodiments, the homodimer is stabilized by a disulfide bond. In some embodiments, the polypeptide shows increased half-life at pH 7.4 compared with a wild type α-GAL polypeptide. In some embodiments, the nucleic acid is a gene therapy construct. In some embodiments, the nucleic acid further comprises a promoter. In some embodiments, the promoter is a constitutive promoter. In some embodiments, the promoter is a tissue-specific promoter. In some embodiments, the nucleic acid comprises at least a portion of a virus nucleic acid sequence. In some embodiments, the virus is selected from wherein the virus comprises a retrovirus, an adenovirus, an adeno associated virus, a lentivirus, or a herpes virus.

Also provided herein are nucleic acid constructs comprising at least one promoter and a nucleic acid encoding a modified α-GAL polypeptide. Modified α-GAL polypeptides are contemplated to comprise cysteine substitutions of an α-GAL polypeptide sequence including: (i) R49C and G361C; (ii) R49C and G360C; (iii) D233C and I359C; (iv) M51C and G360C; and (v) S276C. In some embodiments, the nucleic acid encodes a polypeptide comprising cysteine substitutions of an α-GAL polypeptide sequence selected from the group consisting of: (i) D233C and I359C; and (ii) M51C and G360C. In some embodiments, the nucleic acid encodes a polypeptide comprising cysteine substitutions of an α-GAL polypeptide sequence of D233C and I359C. In some embodiments, the nucleic acid encodes a polypeptide comprising cysteine substitutions of an α-GAL polypeptide sequence of M51C and G360C. In some embodiments, the promoter is a constitutive promoter. In some embodiments, the promoter is a tissue-specific promoter. In some embodiments, the nucleic acid construct comprises at least a portion of a virus nucleic acid sequence. In some embodiments, the virus comprises a retrovirus, an adenovirus, an adeno associated virus, a lentivirus, or a herpes virus. In some embodiments, the polypeptide forms a homodimer. In some embodiments, the homodimer is stabilized by a disulfide bond. In some embodiments, the polypeptide shows increased half-life at pH 7.4 compared with a wild type α-GAL polypeptide. In some embodiments, the nucleic acid is packaged within in a viral capsid protein. In some embodiments, the nucleic acid construct is suitable for gene therapy.

TABLE 2
α-GAL Nucleic Acid Sequences

| α-GAL | | SEQ |
|---|---|---|
| variant | Sequence | ID NO: |
| α-GAL | atgcagctgaggaacccagaactacatctgggctgcgcgcttgcgcttcgcttcctggccacgtacctg | 7 |
| wild | ggacatccctggggctagagcactggacaatggattggcaaggacgcctaccatgggctggctgcactg | |
| type | ggagcgcttcatgtgcaaccttgactgccaggaagagccagattcctgcatcagtgagaagctcttcatgg | |
| | agatggcagagctcatggtctcagaaggctggaaggatgcaggttatgagtacctctgcattgatgactgt | |
| | tggatggctccccaaagagattcagaaggcagacttcaggcagaccctcagcgctttcctcatgggattc | |
| | gccagctagctaattatgttcacagcaaaggactgaagctagggatttatgcagatgttggaaataaaacct | |
| | gcgcaggcttccctgggagttttggatactacgacattgatgcccagacctttgctgactggggagtagat | |
| | ctgctaaaatttgatggttgttactgtgacagtttggaaaatttggcagatggttataagcacatgtccttggc | |
| | cctgaataggactggcagaagcattgtgtactcctgtgagtggcctctttatatgtggccctttcaaaagccc | |
| | aattatacagaaatccgacagtactgcaatcactggcgaaattttgctgacattgatgattcctggaaaagta | |
| | taaagagtatcttggactggacatcttttaaccaggagagaattgttgatgttgctggaccagggggttgga | |
| | atgacccagatatgttagtgattggcaactttggcctcagctggaatcagcaagtaactcagatggccctct | |
| | gggctatcatggctgctcctttattcatgtctaatgacctccgacacatcagccctcaagccaaagctctcct | |
| | tcaggataaggacgtaattgccatcaatcaggaccccttgggcaagcaagggtaccagcttagacaggg | |
| | agacaactttgaagtgtgggaacgacctctctcaggcttagcctgggctgtagctatgataaaccggcag | |
| | gagattggtggacctcgctcttataccatcgcagttgcttccctgggtaaaggagtggcctgtaatcctgcc | |
| | tgcttcatcacacagctcctccctgtgaaaaggaagctagggttctatgaatggacttcaaggttaagaagt | |
| | cacataaatcccacaggcactgttttgcttcagctagaaaatacaatgcagatgtcattaaaagacttacttta | |
| | a | |
| α-GAL | atgcagctgaggaacccagaactacatctgggctgcgcgcttgcgcttcgcttcctggccacgtttcctg | 8 |
| R49C- | ggacatccctggggctagagcactggacaatggattggcaaggacgcctaccatgggctggctgcactg | |
| G361C | ggagTgcttcatgtgcaaccttgactgccaggaagagccagattcctgcatcagtgagaagctcttcatg | |
| | gagatggcagagctcatggtctcagaaggctggaaggatgcaggttatgagtacctctgcattgatgact | |
| | gttggatggctccccaaagagattcagaaggcagacttcaggcagaccacagcgctttcctcatgggatt | |
| | cgccagctagctaattatgttcacagcaaaggactgaagctagggatttatgcagatgttggaaataaaac | |
| | ctgcgcaggcttccctgggagttttggatactacgacattgatgcccagacctttgctgactggggagtag | |
| | atctgctaaaatttgatggttgttactgtgacagtttggaaaatttggcagatggttataagcacatgtccttgg | |
| | ccctgaataggactggcagaagcattgtgtactcctgtgagtggcctctttatatgtggccctttcaaaagcc | |
| | caattatacagaaatccgacagtactgcaatcactggcgaaattttgctgacattgatgattcctggaaaagt | |
| | ataaagagtatcttggactggacatcttttaaccaggagagaattgttgatgttgctggaccagggggttgg | |
| | aatgacccagatatgttagtgattggcaactttggcctcagctggaatcagcaagtaactcagatggccctc | |
| | tgggctatcatggctgctcctttattcatgtctaatgacctccgacacatcagccctcaagccaaagctctcc | |
| | ttcaggataaggacgtaattgccatcaatcaggaccccttgggcaagcaagggtaccagcttagacagg | |
| | gagacaactttgaagtgtgggaacgacctctctcaggcttagcctgggctgtagctatgataaaccggca | |
| | ggagattggtTgCcctcgctcttataccatcgcagttgcttccctgggtaaaggagtggcctgtaatcctg | |
| | cctgcttcatcacacagctcctccctgtgaaaaggaagctagggttctatgaatggacttcaaggttaagaa | |
| | gtcacataaatcccacaggcactgttttgcttcagctagaaaatacaatgcagatgtcattaaaagacttactt | |
| | taa | |
| α-GAL | atgcagctgaggaacccagaactacatctgggctgcgcgcttgcgcttcgcttcctggccctcgtttcctg | 9 |
| R49C- | ggacatccctggggctagagcactggacaatggattggcaaggacgcctaccatgggctggctgcactg | |
| G360C | ggagTgcttcatgtgcaaccttgactgccaggaagagccagattcctgcatcagtgagaagctcttcatg | |
| | gagatggcagagctcatggtctcagaaggctggaaggatgcaggttatgagtacctctgcattgatgact | |
| | gttggatggctccccaaagagattcagaaggcagacttcaggcagaccctcagcgctttcctcatgggatt | |
| | cgccagctagctaattatgttcacagcaaaggactgaagctagggatttatgcagatgttggaaataaaac | |
| | ctgcgcaggcttccctgggagttttggatactacgacattgatgcccagacctttgctgactggggagtag | |
| | atctgctaaaatttgatggttgttactgtgacagtttggaaaatttggcagatggttataagcacatgtccttgg | |
| | ccctgaataggactggcagaagcattgtgtactcctgtgagtggcctctttatatgtggccctttcaaaagcc | |
| | caattatacagaaatccgacagtactgcaatcactggcgaaattttgctgacattgatgattcctggaaaagt | |
| | ataaagagtatcttggactggacatcttttaaccaggagagaattgttgatgttgctggaccagggggttgg | |
| | aatgacccagatatgttagtgattggcaactttggcctcagctggaatcagcaagtaactcagatggccctc | |
| | tgggctatcatggctgctcctttattcatgtctaatgacctccgacacatcagccctcaagccaaagctctcc | |
| | ttcaggataaggacgtaattgccatcaatcaggaccccttgggcaagcaagggtaccagcttagacagg | |
| | gagacaactttgaagtgtgggaacgacctctctcaggcttagcctgggctgtagctatgataaaccggca | |
| | ggagattTgtggacctcgctcttataccatcgcagttgcttccctgggtaaaggagtggcctgtaatcctgc | |
| | ctgcttcatcacacagctcctccctgtgaaaaggaagctagggttctatgaatggacttcaaggttaagaag | |
| | tcacataaatcccacaggcactgttttgcttcagctagaaaatacaatgcagatgtcattaaaagacttacttt | |
| | aa | |
| α-GAL | atgcagctgaggaacccagaactacatctgggctgcgcgcttgcgcttcgcttcctggccctcgtttcctg | 10 |
| M51C- | ggacatccctggggctagagcactggacaatggattggcaaggacgcctaccatgggctggctgcactg | |
| G360C | ggagcgcttcTGCtgcaaccttgactgccaggaagagccagattcctgcatcagtgagaagctcttcat | |
| | ggagatggcagagctcatggtctcagaaggctggaaggatgcaggttatgagtacctctgcattgatgac | |
| | tgttggatggctccccaaagagattcagaaggcagacttcaggcagaccctcagcgctttcctcatgggat | |
| | tcgccagctagctaattatgttcacagcaaaggactgaagctagggatttatgcagatgttggaaataaaac | |
| | ctgcgcaggcttccctgggagttttggatactacgacattgatgcccagacctttgctgactggggagtag | |
| | atctgctaaaatttgatggttgttactgtgacagtttggaaaatttggcagatggttataagcacatgtccttgg | |
| | ccctgaataggactggcagaagcattgtgtactcctgtgagtggcctctttatatgtggccctttcaaaagcc | |
| | caattatacagaaatccgacagtactgcaatcactggcgaaattttgctgacattgatgattcctggaaaagt | |
| | ataaagagtatcttggactggacatcttttaaccaggagagaattgttgatgttgctggaccagggggttgg | |
| | aatgacccagatatgttagtgattggcaactttggcctcagctggaatcagcaagtaactcagatggccctc | |
| | tgggctatcatggctgctcctttattcatgtctaatgacctccgacacatcagccctcaagccaaagctctcc | |
| | ttcaggataaggacgtaattgccatcaatcaggaccccttgggcaagcaagggtaccagcttagacagg | |
| | gagacaactttgaagtgtgggaacgacctctctcaggcttagcctgggctgtagctatgataaaccggca | |
| | ggagattTgtggacctcgctcttataccatcgcagttgcttccctgggtaaaggagtggcctgtaatcctgc | |
| | ctgcttcatcacacagctcctccctgtgaaaaggaagctagggttctatgaatggacttcaaggttaagaag | |
| | tcacataaatcccacaggcactgttttgcttcagctagaaaatacaatgcagatgtcattaaaagacttacttt | |
| | aa | |
| α-GAL | atgcagctgaggaacccagaactacatctgggctgcgcgcttgcgcttcgcttcctggccctcgtttcctg | 11 |
| D233C- | ggacatccctggggctagagcactggacaatggattggcaaggacgcctaccatgggctggctgcactg | |
| I359C | ggagcgcttcatgtgcaaccttgactgccaggaagagccagattcctgcatcagtgagaagctcttcatgg | |
| | agatggcagagctcatggtctcagaaggctggaaggatgcaggttatgagtacctctgcattgatgactgt | |
| | tggatggctccccaaagagattcagaaggcagacttcaggcagaccctcagcgctttcctcatgggattc | |
| | gccagctagctaattatgttcacagcaaaggactgaagctagggatttatgcagatgttggaaataaaacct | |
| | gcgcaggcttccctgggagttttggatactacgacattgatgcccagacctttgctgactggggagtagat | |
| | ctgctaaaatttgatggttgttactgtgacagtttggaaaatttggcagatggttataagcacatgtccttggc | |
| | cctgaataggactggcagaagcattgtgtactcctgtgagtggcctctttatatgtggccctttcaaaagccc | |
| | aattatacagaaatccgacagtactgcaatcactggcgaaattttgctgacattTGCgattcctggaaaa | |
| | gtataaagagtatcttggactggacatcttttaaccaggagagaattgttgatgttgctggaccagggggtt | |
| | ggaatgacccagatatgttagtgattggcaactttggcctcagctggaatcagcaagtaactcagatggcc | |
| | ctctgggctatcatggctgctcctttattcatgtctaatgacctccgacacatcagccctcaagccaaagctc | |
| | tccttcaggataaggacgtaattgccatcaatcaggaccccttgggcaagcaagggtaccagcttagaca | |
| | gggagacaactttgaagtgtgggaacgacctctctcaggcttagcctgggctgtagctatgataaaccgg | |
| | caggagTGCggtggacctcgctcttataccatcgcagttgcttccctgggtaaaggagtggcctgtaat | |
| | cctgcctgcttcatcacacagctcctccctgtgaaaaggaagctagggttctatgaatggacttcaaggtta | |
| | agaagtcacataaatcccacaggcactgttttgcttcagctagaaaatacaatgcagatgtcattaaaagac | |
| | ttactttaa | |
| α-GAL | atgcagctgaggaacccagaactacatctgggctgcgcgcttgcgcttcgcttcctggccctcgtttcctg | 12 |
| S276C | ggacatccctggggctagagcactggacaatggattggcaaggacgcctaccatgggctggctgcactg | |
| | ggagcgcttcatgtgcaaccttgactgccaggaagagccagattcctgcatcagtgagaagctcttcatgg | |
| | agatggcagagctcatggtctcagaaggctggaaggatgcaggttatgagtacctctgcattgatgactgt | |
| | tggatggctccccaaagagattcagaaggcagacttcaggcagaccctcagcgctttcctcatgggattc | |
| | gccagctagctaattatgttcacagcaaaggactgaagctagggatttatgcagatgttggaaataaaacct | |
| | gcgcaggcttccctgggagttttggatactacgacattgatgcccagacctttgctgactggggagtagat | |
| | ctgctaaaatttgatggttgttactgtgacagtttggaaaatttggcagatggttataagcacatgtccttggc | |
| | cctgaataggactggcagaagcattgtgtactcctgtgagtggcctctttatatgtggccctttcaaaagccc | |
| | aattatacagaaatccgacagtactgcaatcactggcgaaattttgctgacattgatgattcctggaaaagta | |
| | taaagagtatcttggactggacatcttttaaccaggagagaattgttgatgttgctggaccagggggttgga | |
| | atgacccagatatgttagtgattggcaactttggcctcTgctggaatcagcaagtaactcagatggccctct | |
| | gggctatcatggctgctcctttattcatgtctaatgacctccgacacatcagccctcaagccaaagctctcct | |
| | tcaggataaggacgtaattgccatcaatcaggaccccttgggcaagcaagggtaccagcttagacaggg | |
| | agacaactttgaagtgtgggaacgacctctctcaggcttagcctgggctgtagctatgataaaccggcag | |
| | gagattggtggacctcgctcttataccatcgcagttgcttccctgggtaaaggagtggcctgtaatcctgcc | |
| | tgcttcatcacacagctcctccctgtgaaaaggaagctagggttctatgaatggacttcaaggttaagaagt | |
| | cacataaatcccacaggcactgttttgcttcagctagaaaatacaatgcagatgtcattaaaagacttacttta | |
| | a | |

In some embodiments, nucleic acids encoding modified α-GAL polypeptides herein have an increased half-life compared with a wild type α-GAL polypeptide. In some embodiments, the half-life is at least 50% greater than a wild type α-GAL polypeptide. In some embodiments, the half-life is at least 150% greater than a wild type α-GAL polypeptide. In some embodiments, the half-life is at least 200% greater than a wild type α-GAL polypeptide. In some embodiments, the half-life is at least 250% greater than a wild type α-GAL polypeptide. In some embodiments, the half-life is at least 300% greater than a wild type α-GAL polypeptide. In some embodiments, the half-life is at least 350% greater than a wild type α-GAL polypeptide.

### Modified PPT-1 Polypeptides

Provided herein are modified PPT-1 polypeptides comprising cysteine substitutions of a PPT-1 polypeptide sequence. Contemplated substitutions provided herein include A171C and A183C. In some embodiments, the polypeptide comprises cysteine substitutions of a PPT-1 polypeptide sequence of A171C and A183C. The modified PPT-1 polypeptide is stabilized by at least one, more preferably two intramolecular disulfide bonds. The modified PPT-1 polypeptides polypeptide show increased half-life at pH 7.4 compared with a wildtype PPT-1 polypeptide.

Wild type and exemplary Modified PPT-1 are provided in Table 3.

TABLE 3
PPT-1 Polypeptide Sequences

| | | SEQ |
|---|---|---|
| PPT-1 | | ID |
| variant | Sequence | NO: |
| Human | MASPGCLWLLAVALLPWTCASRALQHLDPP | 13 |
| PPT-1 | APLPLVIWHGMGDSCCNPLSMGAIKKMVEK | |
| wild | KIPGIYVLSLEIGKTLMEDVENSFFLNVNS | |
| type | QVTTVCQALAKDPKLQQGYNAMGFSQGGQF | |
| (NP_ | LRAVAQRCPSPPMINLISVGGQHQGVFGLP | |
| 000301.1) | RCPGESSHICDFIRKTLNAGAYSKVVQERL | |
| | VQAEYWHDPIKEDVYRNHSIFLADINQERG | |
| | INESYKKNLMALKKFVMVKFLNDSIVDPVD | |
| | SEWFGFYRSGQAKETIPLQETSLYTQDRLG | |
| | LKEMDNAGQLVFLATEGDHLQLSEEWFYAH | |
| | IIPFLG | |
| PPT-1 | MASPGCLWLLAVALLPWTCASRALQHLDPP | 14 |
| A171C | APLPLVIWHGMGDSCCNPLSMGAIKKMVEK | |
| A183C | KIPGIYVLSLEIGKTLMEDVENSFFLNVNS | |
| | QVTTVCQALAKDPKLQQGYNAMGFSQGGQF | |
| | LRAVAQRCPSPPMINLISVGGQHQGVFGLP | |
| | RCPGESSHICDFIRKTLNAGCYSKVVQERL | |
| | VQCEYWHDPIKEDVYRNHSIFLADINQERG | |
| | INESYKKNLMALKKFVMVKFLNDSIVDPVD | |
| | SEWFGFYRSGQAKETIPLQETSLYTQDRLG | |
| | LKEMDNAGQLVFLATEGDHLQLSEEWFYAH | |
| | IIPFLG | |

Also provided herein are modified PPT-1 polypeptides comprising a polypeptide with a sequence containing cysteine residues at positions 171 and 183 and having at least 90% identity to a sequence set forth as SEQ ID NO: 14. In some embodiments, modified PPT-1 polypeptides comprise a polypeptide with a sequence containing cysteine residues at positions 171 and 183 and having at least 95% identity to a sequence set forth as SEQ ID NO: 14. In some embodiments, modified PPT-1 polypeptides comprise a polypeptide with a sequence containing cysteine residues at positions 171 and 183 and having at least 96% identity to a sequence set forth as SEQ ID NO: 14. In some embodiments, modified PPT-1 polypeptides comprise a polypeptide with a sequence containing cysteine residues at positions 171 and 183 and having at least 97% identity to a sequence set forth as SEQ ID NO: 14. In some embodiments, modified PPT-1 polypeptides comprise a polypeptide with a sequence containing cysteine residues at positions 171 and 183 and having at least 98% identity to a sequence set forth as SEQ ID NO: 14. In some embodiments, modified PPT-1 polypeptides comprise a polypeptide with a sequence containing cysteine residues at positions 171 and 183 and having at least 99% identity to a sequence set forth as SEQ ID NO: 14. In some embodiments, modified PPT-1 polypeptides comprise a polypeptide with a sequence containing cysteine residues at positions 171 and 183 and having more than 99% identity to a sequence set forth as SEQ ID NO: 14. In some embodiments, modified PPT-1 polypeptides comprise a polypeptide with a sequence set forth as SEQ ID NO: 14.

In some embodiments, modified PPT-1 polypeptides have an increased half-life at pH 4.6. In some embodiments, the half-life at pH 4.6 is at least 50% greater than a wild type PPT-1 polypeptide. In some embodiments, the half-life at pH 4.6 is at least 150% greater than a wild type PPT-1 polypeptide. In some embodiments, the half-life at pH 4.6 is at least 200% greater than a wild type PPT-1 polypeptide. In some embodiments, the half-life at pH 4.6 is at least 250% greater than a wild type PPT-1 polypeptide. In some embodiments, the half-life at pH 4.6 is at least 300% greater than a wild type PPT-1 polypeptide. In some embodiments, the half-life at pH 4.6 is at least 350% greater than a wild type PPT-1 polypeptide. In some embodiments, the half-life at pH 4.6 is at least 400% greater than a wild type PPT-1 polypeptide.

In some embodiments, the modified PPT-1 polypeptide has a half-life at pH 4.6 that is increased by at least a factor of about 2, 2.5, 3, 3.5, 4, 4.5 or 5 compared to the half-life of wild type PPT-1 at pH 4.6. More preferably, the modified PPT-1 polypeptide has a half-life at pH 4.6 that is increased by at least a factor of about 3, 3.5, or 4 compared to the half-life of wild type PPT-1 polypeptide at pH 4.6.

In some embodiments, the modified PPT-1 polypeptide has an intracellular half-life at that is increased by at least a factor of about 2, 2.5, 3, 3.5, 4, 4.5 or 5 compared to the intracellular half-life of wild type human α-GAL. More preferably, the modified PPT-1 polypeptide has an intracellular half-life that is increased by at least a factor of about 3, 3.5, or 4, 4.5 or 5 compared to the intracellular half-life of wild type PPT-1 polypeptide.

The modified PPT-1 polypeptide has a substantially increased half-life at pH 7.4 compared to wild type human α-GAL.

### Nucleic Acids Encoding Modified PPT-1 Polypeptides

Also provided herein are nucleic acid molecules comprising nucleic acids encoding a modified PPT-1 polypeptide. Contemplated nucleic acids include those encoding a polypeptide comprising cysteine substitutions of a PPT-1 polypeptide sequence including: A171C and A183C. In some embodiments, the nucleic acid encodes a polypeptide comprising cysteine substitutions of A171C and A183C. In some embodiments, the polypeptide is stabilized by a disulfide bond. In some embodiments, the polypeptide shows increased half-life at pH 7.4 compared with a wild type PPT-1 polypeptide. In some embodiments, the nucleic acid is a gene therapy construct. In some embodiments, the nucleic acid further comprises a promoter. In some embodiments, the promoter is a constitutive promoter. In some embodiments, the promoter is a tissue-specific promoter. In some embodiments, the nucleic acid comprises at least a portion of a virus nucleic acid sequence. In some embodiments, the virus is selected from wherein the virus comprises a retrovirus, an adenovirus, an adeno associated virus, a lentivirus, or a herpes virus.

TABLE 4
PT-1 Nucleic Acid Sequences

| | | SEQ |
|---|---|---|
| PPT-1 | | ID |
| variant | Sequence | NO: |
| PPT-1 | TTATTTTGATTCACCGCAGAGGGCGGTCTAC | 15 |
| wild | GAGAGCGCAGAGCCCCACTCGGCCAGCGGGG | |
| type | TCTGGCGGGGGACCTGTCGCGCTGAAAGCTC | |
| | CAGGGTAGGGCCGACGCCCATCAGGCTGGGC | |
| | ATCCGTTCGGGATGCGCAGGTTGCGATCTGC | |
| | AACCGGCGGCGCCACGCCCAGGCGGGCGGAG | |
| | CGCGGTTCCCGGAGTCTCGCGCCCGCGGTCA | |
| | TGTGACACAGCGAAGATGGCGTCGCCCGGCT | |
| | GCCTGTGGCTCTTGGCTGTGGCTCTCCTGCC | |
| | ATGGACCTGCGCTTCTCGGGCGCTGCAGCAT | |
| | CTGGACCCGCCGGCGCCGCTGCCGTTGGTGA | |
| | TCTGGCATGGGATGGGAGACAGCTGTTGCAA | |
| | TCCCTTAAGCATGGGTGCTATTAAAAAAATG | |
| | GTGGAGAAGAAAATACCTGGAATTTACGTCT | |
| | TATCTTTAGAGATTGGGAAGACCCTGATGGA | |
| | GGACGTGGAGAACAGCTTCTTCTTGAATGTC | |
| | AATTCCCAAGTAACAACAGTGTGTCAGGCAC | |
| | TTGCTAAGGATCCTAAATTGCAGCAAGGCTA | |
| | CAATGCTATGGGATTCTCCCAGGGAGGCCAA | |
| | TTTCTGAGGGCAGTGGCTCAGAGATGCCCTT | |
| | CACCTCCCATGATCAATCTGATCTCGGTTGG | |
| | GGGACAACATCAAGGTGTTTTTGGACTCCCT | |
| | CGATGCCCAGGAGAGAGCTCTCACATCTGTG | |
| | ACTTCATCCGAAAAACACTGAATGCTGGGGC | |
| | GTACTCCAAAGTTGTTCAGGAACGCCTCGTG | |
| | CAAGCCGAATACTGGCATGACCCCATAAAGG | |
| | AGGATGTGTATCGCAACCACAGCATCTTCTT | |
| | GGCAGATATAAATCAGGAGCGGGGTATCAAT | |
| | GAGTCCTACAAGAAAAACCTGATGGCCCTGA | |
| | AGAAGTTTGTGATGGTGAAATTCCTCAATGA | |
| | TTCCATTGTGGACCCTGTAGATTCGGAGTGG | |
| | TTTGGATTTTACAGAAGTGGCCAAGCCAAGG | |
| | AAACCATTCCCTTACAGGAGACCTCCCTGTA | |
| | CACACAGGACCGCCTGGGGCTAAAGGAAATG | |
| | GACAATGCAGGACAGCTAGTGTTTCTGGCTA | |
| | CAGAAGGGGACCATCTTCAGTTGTCTGAAGA | |
| | ATGGTTTTATGCCCACATCATACCATTCCTT | |
| | GGATGAAACCCGTATAGTTCACAATAGAGCT | |
| | CAGGGAGCCCCTAACTCTTCCAAACCACATG | |
| | GGAGACAGTTTCCTTCATGCCCAAGCCTGAG | |
| | CTCAGATCCAGCTTGCAACTAATCCTTCTAT | |
| | CATCTAACATGCCCTACTTGGAAAGATCTAA | |
| | GATCTGAATCTTATCCTTTGCCATCTTCTGT | |
| | TACCATATGGTGTTGAATGCAAGTTTAATTA | |
| | CCATGGAGATTGTTTTACAAACTTTTGATGT | |
| | GGTCAAGTTCAGTTTTAGAAAAGGGAGTCTG | |
| | TTCCAGATCAGTGCCAGAACTGTGCCCAGGC | |
| | CCAAAGGAGACAACTAACTAAAGTAGTGAGA | |
| | TAGATTCTAAGGGCAAACATTTTTCCAAGTC | |
| | TTGCCATATTTCAAGCAAAGAGGTGCCCAGG | |
| | CCTGAGGTACTCACATAAATGCTTTGTTTTG | |
| | CTGGTGATTTAACCAGTGCTTGGAAAAATCT | |
| | TGCTTGGCTATTTCTGCATCATTTCTTAAGG | |
| | CTGCCTTCCTCTCTCAGTACGTTGCCCTCTG | |
| | TGCTATCATCTTATCATCAATTATTAGACAA | |
| | ATCCCACTGGCCTACAGTCTTGCTTCTGCAG | |
| | CACCCACTTTGTCTCCTCAGGTAGTGATGAA | |
| | TTAGTTGCTGTCACAAAAGGAGGGAAGTAGC | |
| | ACCCAAATTAAGTTGCTTAAGAGAGGAAATG | |
| | TACATCTTGTATAACTTAGGGAGCGAAGAAA | |
| | ATGTAGGCGCGAAAGTGAAAAGTGAGGCAGC | |
| | TAGTTCTTCCTATTCCATTCTCGACCAACCT | |
| | GCCCTTTCTTAATATGACTAGTGGTCTTGAT | |
| | GCTAGAGTCAACTTACTCTGTTGCTGGCTTT | |
| | AGCAGAGAATAGGAGGAACCATATGAAAAAG | |
| | ATCAGGCTTTCTGACTTCCATCCCCAAAACA | |
| | CATTTACCAGCATACTCCAAACTGTTTCTGA | |
| | TGTGTTCCATGAGAAAAGGATTGTTTGCTCA | |
| | AAAAGCTTGGAAAATACTACACACTCCCTTT | |
| | CTCCTTCTGGAGATCAACCCACATTAGAGTG | |
| | TCTAAGGACTCCTGAGAATTCCTGTTACAGT | |
| | AAACAAAACTAACGTAATCTACCATTTCCTA | |
| | CACTATTTGAGCATGGAAATCATAGTCCCCA | |
| | CTCTGTGAAAACTTAACGCTTTTTGGAAGAC | |
| | ATTTCTGTAGCATGTCAGTTTGGAGAAATGA | |
| | TGAGCTACGCCTTGATGAAAGAACCGTGTTG | |
| | GTGCTGCTAAGTTTAGCCATTATGGTTTTTC | |
| | CTTTCTCTCTCTTAAGCCTTATTCTTCAACT | |
| | AAAAGATGAGGATTAAGAGCAAGAAGTTGGG | |
| | GGGGATGTGAAAATAATTTTATGAGGTTGTC | |
| | TAAAATAAAGAGTAGTTTCTTATC | |
| PPT-1 | ATGGCATCACCGGGTTGCCTCTGGTTGTTGG | 16 |
| A171C | CCGTTGCGTTGCTTCCGTGGACATGTGCATC | |
| A183C | AAGAGCTCTTCAACATCTGGATCCCCCAGCT | |
| | CCCCTGCCGCTCGTAATCTGGCACGGGATGG | |
| | GGGATTCATGTTGTAACCCGTTGTCAATGGG | |
| | CGCGATAAAAAAGATGGTTGAAAAGAAGATT | |
| | CCAGGCATCTACGTTCTGTCCCTGGAAATCG | |
| | GTAAGACACTGATGGAAGACGTGGAGAACTC | |
| | CTTCTTTCTCAACGTCAATAGTCAGGTCACT | |
| | ACCGTCTGTCAAGCATTGGCAAAGGACCCTA | |
| | AACTTCAGCAGGGGTACAATGCGATGGGGTT | |
| | TAGCCAGGGCGGACAGTTTCTTAGAGCCGTC | |
| | GCACAGCGCTGTCCATCTCCCCCGATGATTA | |
| | ACCTTATATCTGTCGGGGGACAACACCAGGG | |
| | TGTTTTTGGTCTTCCTCGCTGTCCTGGTGAA | |
| | AGCTCCCACATCTGTGATTTCATACGCAAAA | |
| | CGTTGAACGCAGGATGCTATAGTAAAGTCGT | |
| | CCAAGAACGGCTTGTTCAATGCGAGTATTGG | |
| | CATGACCCAATAAAAGAAGACGTTTATAGGA | |
| | ATCACTCTATCTTCTTGGCCGATATCAACCA | |
| | AGAACGCGGAATCAACGAAAGCTACAAAAAG | |
| | AATCTTATGGCTCTCAAGAAATTTGTTATGG | |
| | TGAAATTCCTTAATGACTCTATAGTAGATCC | |
| | TGTCGATTCAGAATGGTTCGGGTTCTACAGG | |
| | TCTGGCCAGGCGAAGGAGACTATTCCCCTCC | |
| | AAGAAACGTCTCTCTATACACAAGACAGACT | |
| | CGGACTGAAAGAGATGGATAATGCGGGCCAG | |
| | TTGGTCTTCTTGGCTACGGAAGGCGATCATC | |
| | TCCAACTCTCCGAAGAGTGGTTCTATGCCCA | |
| | TATAATCCCGTTCCTGGGCTAA | |

In some embodiments, nucleic acids encoding modified PPT-1 polypeptides herein have an increased half-life compared with a wild type PPT-1 polypeptide. In some embodiments, the half-life is at least 50% greater than a wild type PPT-1 polypeptide. In some embodiments, the half-life is at least 150% greater than a wild type PPT-1 polypeptide. In some embodiments, the half-life is at least 200% greater than a wild type PPT-1 polypeptide. In some embodiments, the half-life is at least 250% greater than a wild type PPT-1 polypeptide. In some embodiments, the half-life is at least 300% greater than a wild type PPT-1 polypeptide. In some embodiments, the half-life is at least 350% greater than a wild type PPT-1 polypeptide.

### IGF2 Peptides

In some cases, modified polypeptides herein, such as modified α-GAL or modified PPT-1 polypeptides herein are fused to an Insulin-Like Growth Factor 2 (IGF2) peptide for targeting modified polypeptides to the lysosome where they are needed. Variants in the IGF2 peptide sequence maintain high affinity binding to IGF2/CI-MPR and eliminate binding to IGF1, insulin receptors, and IGF binding proteins (IGFBP). The variant IGF2 peptide is substantially more selective and has reduced safety risks compared to conventional IGF2 fusion proteins. IGF2 peptides herein include those having an amino acid sequence of AYRPSETLCGGELVDTLQFVCGDRGFYFSRPASRVSRRSRGIVEECCFRSCDLALLETYCATPAKSE (SEQ ID NO: 17). Additional IGF2 peptides have variant amino acid sequences optimized for improved targeting. Variant IGF2 peptides include variant amino acids at positions, 26, 27, 43, 48, 49, 50, 54, 55, or 65 of a wild type IGF2 sequence. These include substitutions at F26, Y27, V43, F48, R49, 550, A54, L55, or K65 of SEQ ID NO: 17. In some embodiments, the IGF2 peptide has a sequence having one or more substitutions from the group consisting of F26S, Y27L, V43L, F48T, R49S, S50I, A54R, L55R, and K65R. In some embodiments, the IGF2 peptide has a sequence having a substitution of F26S. In some embodiments, the IGF2 peptide has a sequence having a substitution of Y27L. In some embodiments, the IGF2 peptide has a sequence having a substitution of V43L. In some embodiments, the IGF2 peptide has a sequence having a substitution of F48T. In some embodiments, the IGF2 peptide has a sequence having a substitution of R49S. In some embodiments, the IGF2 peptide has a sequence having a substitution of S50I. In some embodiments, the IGF2 peptide has a sequence having a substitution of A54R. In some embodiments, the IGF2 peptide has a sequence having a substitution of L55R. In some embodiments, the IGF2 peptide has a sequence having a substitution of K65R. In some embodiments, the IGF2 peptide has a sequence having a substitution of F26S, Y27L, V43L, F48T, R49S, S50I, A54R, and L55R. In some embodiments, the IGF2 peptide has an N-terminal deletion. In some embodiments, the IGF2 peptide has an N-terminal deletion of one amino acid. In some embodiments, the IGF2 peptide has an N-terminal deletion of two amino acids. In some embodiments, the IGF2 peptide has an N-terminal deletion of three amino acids. In some embodiments, the IGF2 peptide has an N-terminal deletion of three amino acids. In some embodiments, the IGF2 peptide has an N-terminal deletion of four amino acids. In some embodiments, the IGF2 peptide has an N-terminal deletion of five amino acids. In some embodiments, the IGF2 peptide has an N-terminal deletion of six amino acids. In some embodiments, the IGF2 peptide has an N-terminal deletion of seven amino acids. In some embodiments, the IGF2 peptide has an N-terminal deletion of seven amino acids and a substitution of Y27L and K65R.

Additional substitutions are contemplated for decreasing instability while maintaining CI-MPR binding affinity. These substitutions are contemplated to be combined with any other substitution described herein. In some embodiments, the IGF2 peptide has a sequence having a substitution of L17N. In some embodiments, the IGF2 peptide has a sequence having a substitution of P31G. In some embodiments, the IGF2 peptide has a sequence having a substitution of R38G. In some embodiments, the IGF2 peptide has a sequence having a substitution of E45W. In some embodiments, the IGF2 peptide has a sequence having a substitution of S50G. In some embodiments, the IGF2 peptide has a sequence having substitutions of R38G and E45W. In some embodiments, the IGF2 peptide has a sequence having substitutions of R38G, E45W, and S50G. In some embodiments, the IGF2 peptide has a sequence having substitutions of P31G, R38G, E45W, and S50G. In some embodiments, the IGF2 peptide has a sequence having substitutions of L17N, P31G, R38G, E45W, and S50G. Exemplary peptide sequences are represented by SEQ ID NOs: 17-27.

TABLE 5
IGF Peptide Sequences (variant residues
are underlined)

| | | SEQ |
|---|---|---|
| | | ID |
| Peptide | Sequence | NO |
| Wild | AYRPSETLCGGELVDTLQFVCGDRGFYFSRPASR | 17 |
| type | VSRRSRGIVEECCFRSCDLALLETYCATPAKSE | |
| F26S | AYRPSETLCGGELVDTLQFVCGDRGSYFSRPASR | 18 |
| | VSRRSRGIVEECCFRSCDLALLETYCATPAKSE | |
| Y27L | AYRPSETLCGGELVDTLQFVCGDRGFLFSRPASRV | 19 |
| | SRRSRGIVEECCFRSCDLALLETYCATPAKSE | |
| V43L | AYRPSETLCGGELVDTLQFVCGDRGFYFSRPASR | 20 |
| | VSRRSRGILEECCFRSCDLALLETYCATPAKSE | |
| F48T | AYRPSETLCGGELVDTLQFVCGDRGFYFSRPASR | 21 |
| | VSRRSRGIVEECCTRSCDLALLETYCATPAKSE | |
| R49S | AYRPSETLCGGELVDTLQFVCGDRGFYFSRPASR | 22 |
| | VSRRSRGIVEECCFSSCDLALLETYCATPAKSE | |
| S50I | AYRPSETLCGGELVDTLQFVCGDRGFYFSRPASR | 23 |
| | VSRRSRGIVEECCFRICDLALLETYCATPAKSE | |
| A54R | AYRPSETLCGGELVDTLQFVCGDRGFYFSRPASR | 24 |
| | VSRRSRGIVEECCFRSCDLRLLETYCATPAKSE | |
| L55R | AYRPSETLCGGELVDTLQFVCGDRGFYFSRPASR | 25 |
| | VSRRSRGIVEECCFRSCDLARLETYCATPAKSE | |
| F26S, | AYRPSETLCGGELVDTLQFVCGDRGSLFSRPASRV | 26 |
| Y27L, | SRRSRGILEECCTSICDLRRLETYCATPAKSE | |
| V43L, | | |
| F48T, | | |
| R49S, | | |
| S50I, | | |
| A54R, | | |
| L55R | | |
| Δ1-6, | TLCGGELVDTLQFVCGDRGFLFSRPASRVSRRSRG | 27 |
| Y27L, | IVEECCFRSCDLALLETYCATPARSE | |
| K65R | | |

### Internal Ribosomal Entry Sequences

Nucleic acids encoding a modified polypeptides herein, such as nucleic acids encoding modified α-GAL and PP-1 polypeptides, in some embodiments, further comprise an internal ribosome entry sequence (IRES) for increasing gene expression by bypassing the bottleneck of translation initiation. Suitable internal ribosomal entry sequences for optimizing expression for gene therapy include but are not limited to a cricket paralysis virus (CrPV) IRES, a picomavirus IRES, an Aphthovirus IRES, a Kaposi's sarcoma-associated herpesvirus IRES, a Hepatitis A IRES, a Hepatitis C IRES, a Pestivirus IRES, a Cripavirus IRES, a Rhopalosiphum padi virus IRES, a Merek's disease virus IRES, and other suitable IRES sequences. In some embodiments, the gene therapy construct comprises a CrPV IRES. In some embodiments, the CrPV IRES has a nucleic acid sequence of

(SEQ ID NO: 28)
CGGUGUCGAAGUAGAAUUUCUAUCUCGACACGCGGCCUUCCAAGCAGUUAG
GGAAACCGACUUCUUUGAAGAAGAAAGCUGACUAUGUGAUCUUAUUAAAAU
UAGGUUAAAUUUCGAGGUUAAAAAUAGUUUUAAUAUUGCUAUAGUCUUAGA
GGUCUUGUAUAUUUAUACUUACCACACAAGAUGGACCGGAGCAGCCCUCCA
AUAUCUAGUGUACCCUCGUGCUCGCUCAAACAUUAAGUGGUGUUGUGCGAA
AAGAAUCUCACUUCAAGAA

### Signal Sequence

Provided herein are nucleic acid molecules comprising nucleic acids encoding modified polypeptides, such as modified α-GAL polypeptides or modified PPT-1 polypeptides, wherein the nucleic acid molecules further comprise a signal peptide, which improves secretion of the therapeutic protein from the cell transduced with the gene therapy construct. The signal peptide in some embodiments improves protein processing of therapeutic proteins, and facilitates translocation of the nascent polypeptide-ribosome complex to the ER and ensuring proper co-translational and post-translational modifications. In some embodiments, the signal peptide is located (i) in an upstream position of the signal translation initiation sequence, (ii) in between the translation initiation sequence and the therapeutic protein, or (iii) a downstream position of the therapeutic protein. Signal peptides useful in gene therapy constructs include but are not limited to binding immunoglobulin protein (BiP) signal peptide from the family of HSP70 proteins (e.g., HSPA5, heat shock protein family A member 5), and variants thereof. These signal peptides have ultrahigh affinity to the signal recognition particle. Examples of BiP amino acid sequences are provided in Table 6 below. In some embodiments, the signal peptide has an amino acid sequence that is at least 90% identical to a sequence selected from the group consisting of SEQ ID Nos: 29-33. In some embodiments, the signal peptide differs from a sequence selected from the group consisting of SEQ ID Nos: 29-33 by 5 or fewer, 4 or fewer, 3 or fewer, 2 or fewer, or 1 amino acid.

TABLE 6
Signal Sequences

| | | SEQ |
|---|---|---|
| Signal | | ID |
| Sequence | Amino Acid Sequence | NO: |
| Native human | MKLSLVAAMLLLLSAARA | 29 |
| Bip | | |
| Modified Bip-1 | MKLSLVAAMLLLLSLVAAMLLLLSAARA | 30 |
| Modified Bip-2 | MKLSLVAAMLLLLWVALLLLSAARA | 31 |
| Modified Bip-3 | MKLSLVAAMLLLLSLVALLLLSAARA | 32 |
| Modified Bip-4 | MKLSLVAAMLLLLALVALLLLSAARA | 33 |

### Kozak Sequence

Provided herein are nucleic acid molecules comprising nucleic acids encoding modified polypeptides, such as modified α-GAL polypeptides or modified PPT-1 polypeptides, wherein the nucleic acid molecules further comprise a nucleic acid having a kozak sequence, which aids in initiation of translation of the mRNA. Kozak sequences contemplated herein have a consensus sequence of gccRccAUGG (SEQ ID NO: 34) where a lowercase letter denotes the most common base at the position and the base varies, uppercase letters indicate highly conserved bases that only vary rarely change. R indicates that a purine (adenine or guanine) is always observed at that position. The sequence in parentheses (gcc) is of uncertain significance.

### Therapeutic Protein

Gene therapy constructs provided herein comprise a nucleic acid encoding a stabilized form of a protein for treating a genetic disorder. The therapeutic protein expressed from the gene therapy construct replaces the absent or defective protein. Therapeutic proteins, therefore, are chosen based on the genetic defect in need of treatment in an individual. Stabilized forms herein comprise one or more non-native cysteine residues that form a disulfide bridge between the non-native cysteines within the protein or between non-native cysteines of two monomers of the protein.

In some embodiments, gene therapy constructs herein encode an enzyme, such as an enzyme having a genetic defect in an individual with a lysosomal storage disorder. In some embodiments, enzymes encoded by gene therapy constructs provided herein include but are not limited to alpha-galactosidase A, β-glucocerebrosidase, glucocerebrosidase, lysosomal acid lipase, glycosaminoglycan alpha-L-iduronidase, alpha-L-iduronidase, N-sulfoglucosamine sulfohydrolase (SGSH), N-acetyl-alpha-glucosaminidase (NAGLU), iduronate-2-sulfatase, N-acetylgalactosamine-6-sulfatase, glycosaminoglycan N-acetylgalactosamine 4-sulfatase, alpha-glucosidase, tripeptidyl peptidase 1 (TPP1), palmitoyl protein thioesterases, ceroid lipofuscinoses neuronal 1, ceroid lipofuscinoses neuronal 2, ceroid lipofuscinoses neuronal 3, ceroid lipofuscinoses neuronal 4, ceroid lipofuscinoses neuronal 5, ceroid lipofuscinoses neuronal 6, ceroid lipofuscinoses neuronal 7, ceroid lipofuscinoses neuronal 8, ceroid lipofuscinoses neuronal 9, ceroid lipofuscinoses neuronal 10, ceroid lipofuscinoses neuronal 11, ceroid lipofuscinoses neuronal 12, ceroid lipofuscinoses neuronal 13, ceroid lipofuscinoses neuronal 14, ceroid lipofuscinoses neuronal 15, ceroid lipofuscinoses neuronal 16, and cyclin dependent kinase like 5.

### Gene Therapy Vectors and Compositions

Provided herein are gene therapy vectors comprising a nucleic acid construct comprising: a nucleic acid encoding a stabilized form of a protein for treating a neurological or genetic disorder, the stabilized form comprising one or more non-native cysteine residues that form a disulfide bridge between non-native cysteines within the protein or between non-native cysteines of two monomers of the protein. In some embodiments, the stabilized form comprises a modified α-GAL polypeptide or a modified PPT-1 polypeptide.

In some embodiments, the nucleic acid encoding a modified polypeptide is cloned into a number of types of vectors. For example, in some embodiments, the nucleic acid is cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector encoding the modified polypeptide is provided to a cell in the form of a viral vector. Viral vector technology is described, e.g., in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

Also provided herein are compositions and systems for gene transfer. A number of virally based systems have been developed for gene transfer into mammalian cells. For example, retroviruses provide a convenient platform for gene delivery systems. A selected gene, in some embodiments, is inserted into a vector and packaged in retroviral particles using suitable techniques. The recombinant virus is then isolated and delivered to cells of the subject either in vivo or ex vivo. A number of retroviral systems are suitable for gene therapy. In some embodiments, adenovirus vectors are used. A number of adenovirus vectors are suitable for gene therapy. In some embodiments, adeno-associated virus vectors are used. A number of adeno-associated viruses are suitable for gene therapy. In one embodiment, lentivirus vectors are used.

Gene therapy constructs provided herein comprise a vector (or gene therapy expression vector) into which the gene of interest is cloned or otherwise which includes the gene of interest in a manner such that the nucleotide sequences of the vector allow for the expression (constitutive or otherwise regulated in some manner) of the gene of interest. The vector constructs provided herein include any suitable gene expression vector that is capable of being delivered to a tissue of interest and which will provide for the expression of the gene of interest in the selected tissue of interest.

In some embodiments, the vector is an adeno-associated virus (AAV) vector because of the capacity of AAV vectors to cross the blood-brain barrier and transduction of neuronal tissue. In methods provided herein, AAV of any serotype is contemplated to be used. The serotype of the viral vector used in certain embodiments is selected from the group consisting of AAV1 vector, an AAV2 vector, an AAV3 vector, an AAV4 vector, an AAV5 vector, an AAV6 vector, an AAV7 vector, an AAV8 vector, an AAV9 vector, an AAVrhS vector, an AAVrh10 vector, an AAVrh33 vector, an AAVrh34 vector, an AAVrh74 vector, an AAV Anc80 vector, an AAVPHP.B vector, an AAVhu68 vector, an AAV-DJ vector, and others suitable for gene therapy.

AAV vectors are DNA parvoviruses that are nonpathogenic for mammals. Briefly, AAV-based vectors have the rep and cap viral genes that account for 96% of the viral genome removed, leaving the two flanking 145 base pair inverted terminal repeats (ITR) which are used to initiate viral DNA replication, packaging, and integration.

Further embodiments include use of other serotype capsids to create an AAV1 vector, an AAV2 vector, an AAV3 vector, an AAV4 vector, an AAV5 vector, an AAV6 vector, an AAV7 vector, an AAV8 vector, an AAV9 vector, an AAVrhS vector, an AAVrh10 vector, an AAVrh33 vector, an AAVrh34 vector, an AAVrh74 vector, an AAV Anc80 vector, an AAVPHP.B vector, an AAV-DJ vector, and others suitable for gene therapy. Optionally, the AAV viral capsid is AAV2/9, AAV9, AAVrhS, AAVrh10, AAVAnc80, or AAV PHP.B.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements is often increased to 50 bp apart before activity begins to decline. Depending on the promoter, it appears that individual elements function either cooperatively or independently to activate transcription.

An example of a promoter that is capable of expressing a stabilized protein, such as a modified α-GAL polypeptide or a modified PPT-1 polypeptide transgene in a mammalian T-cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving expression from transgenes cloned into a lentiviral vector (see, e.g., Milone et al., Mol. Ther. 17(8): 1453-1464 (2009)). Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences are sometimes also used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, gene therapy vectors are not contemplated to be limited to the use of constitutive promoters. Inducible promoters are also contemplated here. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline-regulated promoter. In some embodiments, the promoter is an α-GAL promoter.

In order to assess the expression of a modified polypeptide the expression vector to be introduced into a cell often contains either a selectable marker gene or a reporter gene or both to facilitate identification and selection of expressing cells from the population of cells sought to be transfected or infected through viral vectors. In other aspects, the selectable marker is often carried on a separate piece of DNA and used in a co-transfection procedure. Both selectable markers and reporter genes are sometimes flanked with appropriate regulatory sequences to enable expression in the host cells. Useful selectable markers include, for example, antibiotic-resistance genes, such as neo and the like.

Methods of introducing and expressing genes into a cell are suitable for methods herein. In the context of an expression vector, the vector is readily introduced into a host cell, e.g., mammalian, bacterial, yeast, or insect cell by any method in the art. For example, the expression vector is transferred into a host cell by physical, chemical, or biological means.

Physical methods for introducing a polynucleotide into a host cell include calcium phosphate precipitation, lipofection, particle bombardment, microinjection, gene gun, electroporation, and the like. Methods for producing cells comprising vectors and/or exogenous nucleic acids are suitable for methods herein (see, e.g., Sambrook et al., 2012, Molecular Cloning: A Laboratory Manual, volumes 1-4, Cold Spring Harbor Press, NY). One method for the introduction of a polynucleotide into a host cell is calcium phosphate transfection

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid is associated with a lipid. The nucleic acid associated with a lipid, in some embodiments, is encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some embodiments, liposomes are present in a bilayer structure, as micelles, or with a “collapsed” structure. Alternately, liposomes are simply interspersed in a solution, possibly forming aggregates that are not uniform in size or shape. Lipids are fatty substances which are, in some embodiments, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. For example, in some embodiments, dimyristyl phosphatidylcholine (“DMPC”) is obtained from Sigma, St. Louis, Mo.; in some embodiments, dicetyl phosphate (“DCP”) is obtained from K & K Laboratories (Plainview, N.Y.); cholesterol (“Choi”), in some embodiments, is obtained from Calbiochem-Behring; dimyristyl phosphatidylglycerol (“DMPG”) and other lipids are often obtained from Avanti Polar Lipids, Inc. (Birmingham. Ala.). Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids, in some embodiments, assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the a modified α-GAL polypeptide in order to confirm the presence of the recombinant DNA sequence in the host cell, a variety of assays are contemplated to be performed. Such assays include, for example, “molecular biological” assays suitable for methods herein, such as Southern and Northern blotting, RT-PCR and PCR; “biochemical” assays, such as detecting the presence or absence of a particular peptide, e.g., by immunological means (ELISAs and western blots) or by assays described herein to identify agents falling within the scope herein.

The present disclosure further provides a vector comprising a modified polypeptide encoding nucleic acid molecule. In one aspect, a therapeutic fusion protein vector is capable of being directly transduced into a cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the modified polypeptide construct in mammalian cells. In one aspect, the mammalian cell is a human cell.

### Pharmaceutical Compositions

Provided herein are pharmaceutical compositions comprising a modified polypeptide a stabilized form of a protein for treating a genetic disorder, the stabilized form comprising one or more non-native cysteine residues that form a disulfide bridge between non-native cysteines within the protein or between non-native cysteines of two monomers of the protein and (ii) a pharmaceutically acceptable excipient. In some embodiments, the modified polypeptide forms a homodimer. In some embodiments, the homodimer is stabilized by a disulfide bond. In some embodiments, the modified polypeptide shows increased half-life at pH 7.4 compared with a wild type polypeptide.

Additionally provided herein are pharmaceutical compositions comprising (i) a modified α-GAL polypeptide, wherein the modified α-GAL polypeptide comprises cysteine substitutions of an α-GAL polypeptide sequence and (ii) a pharmaceutically acceptable excipient. Contemplated substitutions include: (i) D233C and I359C; and (ii) M51C and G360C. In some embodiments, the modified α-GAL polypeptide comprises cysteine substitutions of an α-GAL polypeptide sequence of D233C and I359C. In some embodiments, the modified α-GAL polypeptide comprises cysteine substitutions of an α-GAL polypeptide sequence of M51C and G360C. In some embodiments, the modified α-GAL polypeptide forms a homodimer. In some embodiments, the homodimer is stabilized by a disulfide bond. In some embodiments, the modified α-GAL polypeptide shows increased half-life at pH 7.4 compared with a wild type α-GAL polypeptide. In some embodiments, the composition comprises a chaperone. In some embodiments, the chaperone comprises Migalastat.

Further provided herein are pharmaceutical compositions comprising (i) a modified PPT-1 polypeptide, wherein the modified PPT-1 polypeptide comprises cysteine substitutions of a PPT-1 polypeptide sequence and (ii) a pharmaceutically acceptable excipient. Contemplated substitutions include A171C and A183C. In some embodiments, the modified PPT-1 polypeptide forms a homodimer. In some embodiments, the homodimer is stabilized by a disulfide bond. In some embodiments, the modified PPT-1 polypeptide shows increased half-life at pH 7.4 compared with a wild type PPT-1 polypeptide. In some embodiments, the composition comprises a chaperone.

Suitable excipients for pharmaceutical compositions herein include but are not limited to saline, maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate.

In some embodiments, pharmaceutical compositions herein comprise modified α-GAL polypeptides herein having an increased half-life at pH 4.6 compared with a wild type α-GAL polypeptide. In some embodiments, the half-life at pH 4.6 is at least 50% greater than a wild type α-GAL polypeptide. In some embodiments, the half-life at pH 4.6 is at least 150% greater than a wild type α-GAL polypeptide. In some embodiments, the half-life at pH 4.6 is at least 200% greater than a wild type α-GAL polypeptide. In some embodiments, the half-life at pH 4.6 is at least 250% greater than a wild type α-GAL polypeptide. In some embodiments, the half-life at pH 4.6 is at least 300% greater than a wild type α-GAL polypeptide. In some embodiments, the half-life at pH 4.6 is at least 350% greater than a wild type α-GAL polypeptide. In some embodiments, the half-life at pH 4.6 is at least 400% greater than a wild type α-GAL polypeptide.

In some embodiments, pharmaceutical compositions herein comprise modified PPT-1 polypeptides herein having an increased half-life at pH 4.6 compared with a wild type PPT-1 polypeptide. In some embodiments, the half-life at pH 4.6 is at least 50% greater than a wild type PPT-1 polypeptide. In some embodiments, the half-life at pH 4.6 is at least 150% greater than a wild type PPT-1 polypeptide. In some embodiments, the half-life at pH 4.6 is at least 200% greater than a wild type PPT-1 polypeptide. In some embodiments, the half-life at pH 4.6 is at least 250% greater than a wild type PPT-1 polypeptide. In some embodiments, the half-life at pH 4.6 is at least 300% greater than a wild type PPT-1 polypeptide. In some embodiments, the half-life at pH 4.6 is at least 350% greater than a wild type PPT-1 polypeptide. In some embodiments, the half-life at pH 4.6 is at least 400% greater than a wild type PPT-1 polypeptide.

In some embodiments, pharmaceutical compositions herein comprise modified α-GAL polypeptides having an increased half-life at pH 7.4. In some embodiments, the half-life at pH 7.4. is at least 50% greater than a wild type α-GAL polypeptide. In some embodiments, the half-life at pH 7.4 is at least 150% greater than a wild type α-GAL polypeptide. In some embodiments, the half-life at pH 7.4 is at least 200% greater than a wild type α-GAL polypeptide. In some embodiments, the half-life at pH 7.4 is at least 250% greater than a wild type α-GAL polypeptide. In some embodiments, the half-life at pH 7.4 is at least 300% greater than a wild type α-GAL polypeptide. In some embodiments, the half-life at pH 7.4 is at least 350% greater than a wild type α-GAL polypeptide. In some embodiments, the half-life at pH 7.4 is at least 400% greater than a wild type α-GAL polypeptide. In some embodiments, the half-life at pH 7.4 is at least 500% greater than a wild type α-GAL polypeptide. In some embodiments, the half-life at pH 7.4 is at least 600% greater than a wild type α-GAL polypeptide. In some embodiments, the half-life at pH 7.4 is at least 700% greater than a wild type α-GAL polypeptide. In some embodiments, the half-life at pH 7.4 is at least 800% greater than a wild type α-GAL polypeptide. In some embodiments, the half-life at pH 7.4 is at least 900% greater than a wild type α-GAL polypeptide. In some embodiments, the half-life at pH 7.4 is at least 1000% greater than a wild type α-GAL polypeptide.

In some embodiments, pharmaceutical compositions herein comprise modified PPT-1 polypeptides having an increased half-life at pH 7.4. In some embodiments, the half-life at pH 7.4. is at least 50% greater than a wild type PPT-1 polypeptide. In some embodiments, the half-life at pH 7.4 is at least 150% greater than a wild type PPT-1 polypeptide. In some embodiments, the half-life at pH 7.4 is at least 200% greater than a wild type PPT-1 polypeptide. In some embodiments, the half-life at pH 7.4 is at least 250% greater than a wild type PPT-1 polypeptide. In some embodiments, the half-life at pH 7.4 is at least 300% greater than a wild type PPT-1 polypeptide. In some embodiments, the half-life at pH 7.4 is at least 350% greater than a wild type PPT-1 polypeptide. In some embodiments, the half-life at pH 7.4 is at least 400% greater than a wild type PPT-1 polypeptide. In some embodiments, the half-life at pH 7.4 is at least 500% greater than a wild type PPT-1 polypeptide. In some embodiments, the half-life at pH 7.4 is at least 600% greater than a wild type PPT-1 polypeptide. In some embodiments, the half-life at pH 7.4 is at least 700% greater than a wild type PPT-1 polypeptide. In some embodiments, the half-life at pH 7.4 is at least 800% greater than a wild type PPT-1 polypeptide. In some embodiments, the half-life at pH 7.4 is at least 900% greater than a wild type PPT-1 polypeptide. In some embodiments, the half-life at pH 7.4 is at least 1000% greater than a wild type PPT-1 polypeptide.

### Methods of Treatment

### Gene Therapy Methods

Also provided herein are methods of ameliorating at least one symptom of a genetic disease in a subject in need thereof. Some such methods comprise administering at least one dose of a composition comprising a gene therapy a nucleic acid encoding a stabilized form of a protein for treating a genetic disorder, the stabilized form comprising one or more non-native cysteine residues that form a disulfide bridge between non-native cysteines within the protein or between non-native cysteines of two monomers of the protein. In some embodiments, the nucleic acid encodes a polypeptide which forms a homodimer. In some embodiments, the homodimer is stabilized by a disulfide bond. In some embodiments, the nucleic acid encodes a polypeptide having increased half-life at pH 7.4 compared with a wild type polypeptide. In some embodiments, the promoter is a constitutive promoter. In some embodiments, the promoter is a tissue-specific promoter. In some embodiments, the nucleic acid comprises at least a portion of a virus. In some embodiments, the virus is selected from wherein the virus comprises a retrovirus, an adenovirus, an adeno associated virus, a lentivirus, or a herpes virus. In some embodiments, the nucleic acid is packaged within in a viral capsid protein. In some embodiments, the at least one symptom is selected from one or more of pain, skin discoloration, inability to sweat, eye cloudiness, gastrointestinal dysfunction, tinnitus, hearing loss, mitral valve prolapse, heart disease, joint pain, renal failure, and kidney dysfunction. In some embodiments, at least one symptom is reduced with a single administration of the gene therapy nucleic acid construct. In some embodiments, the method further comprises measuring an activity in a tissue obtained from the subject following treatment.

In some embodiments the gene therapy vector or pharmaceutical composition is administered to the cerebrospinal fluid. In some embodiments, the gene therapy vector or pharmaceutical composition is delivered by intrathecal, intracerebroventricular, intraperenchymal, or intravenous injection, or a combination thereof. In some embodiments, the gene therapy vector or pharmaceutical composition is administered by intrathecal injection. In some embodiments, the gene therapy vector or pharmaceutical composition is administered via intravenous injection.

In some embodiments, the genetic disorder is a neurological disorder. In some embodiments, the genetic disorder is a lysosomal storage disorder. In some embodiments, genetic disorder is selected from the group consisting of aspartylglucosaminuria, Batten disease, cystinosis, Fabry disease, Gaucher disease type I, Gaucher disease type II, Gaucher disease type III, Pompe disease, Tay Sachs disease, Sandhoff disease, metachomatic leukodystrophy, mucolipidosis type I, mucolipidosis type II, mucolipidosis type III, mucolipidosis type IV, Hurler disease, Hunter disease, Sanfilippo disease type A, Sanfilippo disease type B, Sanfilippo disease type C, Sanfilippo disease type D, Morquio disease type A, Morquio disease type B, Maroteau-Lamy disease, Sly disease, Niemann-Pick disease type A, Niemann-Pick disease type B, Niemann-Pick disease type C1, Niemann-Pick disease type C2, Schindler disease type I, Schindler disease type II, adenosine deaminase severe combined immunodeficiency (ADA-SCID), chronic granulomatous disease (CGD), infantile, juvenile and adult forms of neuronal ceroid lipofuscinosis, and CDKL5 deficiency disease.

Also provided herein are methods of ameliorating at least one symptom of Fabry disease in a subject in need thereof. Some such methods comprise administering at least one dose of a composition comprising a gene therapy nucleic acid construct comprising at least one promoter and a nucleic acid encoding a modified α-GAL polypeptide comprising cysteine substitutions of an α-GAL polypeptide sequence. Modified α-GAL polypeptides are contemplated to comprise cysteine substitutions including: (i) R49C and G361C; (ii) R49C and G360C; (iii) D233C and I359C; (iv) M51C and G360C; and (v) S276C. In some embodiments, the nucleic acid encodes a polypeptide comprising cysteine substitutions of an α-GAL polypeptide sequence selected from the group consisting of: (i) D233C and I359C; and (ii) M51C and G360C. In some embodiments, the nucleic acid encodes a polypeptide comprising cysteine substitutions of an α-GAL polypeptide sequence of D233C and I359C. In some embodiments, the nucleic acid encodes a polypeptide comprising cysteine substitutions of an α-GAL polypeptide sequence of M51C and G360C. In some embodiments, the nucleic acid encodes a polypeptide which forms a homodimer. In some embodiments, the homodimer is stabilized by a disulfide bond. In some embodiments, the nucleic acid encodes a modified α-GAL polypeptide having increased half-life at pH 7.4 compared with a wild type α-GAL polypeptide. In some embodiments, the promoter is a constitutive promoter. In some embodiments, the promoter is a tissue-specific promoter. In some embodiments, the nucleic acid comprises at least a portion of a virus. In some embodiments, the virus is selected from wherein the virus comprises a retrovirus, an adenovirus, an adeno associated virus, a lentivirus, or a herpes virus. In some embodiments, the nucleic acid is packaged within in a viral capsid protein. In some embodiments, the at least one symptom is selected from one or more of pain, skin discoloration, inability to sweat, eye cloudiness, gastrointestinal dysfunction, tinnitus, hearing loss, mitral valve prolapse, heart disease, joint pain, renal failure, and kidney dysfunction. In some embodiments, at least one symptom is reduced with a single administration of the gene therapy nucleic acid construct. In some embodiments, the method further comprises measuring an α-GAL activity in a tissue obtained from the subject following treatment. In some embodiments, the method further comprises administering a chaperone. In some embodiments, the chaperone comprises Migalastat.

Also provided herein are methods of ameliorating at least one symptom of CLN1 disease in a subject in need thereof. Some such methods comprise administering at least one dose of a composition comprising a gene therapy nucleic acid construct comprising at least one promoter and a nucleic acid encoding a modified PPT-1 polypeptide comprising cysteine substitutions of PPT-1 polypeptide sequence. Modified PPT-1 polypeptides are contemplated to comprise cysteine substitutions including A171C and A183C. In some embodiments, the nucleic acid encodes a polypeptide which forms a homodimer. In some embodiments, the homodimer is stabilized by a disulfide bond. In some embodiments, the nucleic acid encodes a modified PPT-1 polypeptide having increased half-life at pH 7.4 compared with a wild type PPT-1 polypeptide. In some embodiments, the promoter is a constitutive promoter. In some embodiments, the promoter is a tissue-specific promoter. In some embodiments, the nucleic acid comprises at least a portion of a virus. In some embodiments, the virus is selected from wherein the virus comprises a retrovirus, an adenovirus, an adeno associated virus, a lentivirus, or a herpes virus. In some embodiments, the nucleic acid is packaged within in a viral capsid protein. In some embodiments, the at least one symptom is selected from one or more of pain, skin discoloration, inability to sweat, eye cloudiness, gastrointestinal dysfunction, tinnitus, hearing loss, mitral valve prolapse, heart disease, joint pain, renal failure, and kidney dysfunction. In some embodiments, at least one symptom is reduced with a single administration of the gene therapy nucleic acid construct. In some embodiments, the method further comprises measuring a PPT-1 activity in a tissue obtained from the subject following treatment. In some embodiments, the method further comprises administering a chaperone. In some embodiments, the chaperone comprises Migalastat.

In some embodiments, treatment via methods described herein delivers a gene encoding a therapeutic protein to a cell in need of the therapeutic protein. In some embodiments, the treatment delivers the gene to all somatic cells in the individual. In some embodiments, the treatment replaces the defective gene in the targeted cells. In some embodiments, cells treated ex vivo to express the therapeutic protein are delivered to the individual.

In some embodiments, gene therapy treatments herein comprise administering a nucleic acid encoding modified α-GAL polypeptides herein having an intracellular half-life that is increased by at least a factor of about 2, 2.5, 3, 3.5, 4, 4.5 or 5 compared to the half-life of wild type human α-GAL.

### Enzyme Replacement Therapy Methods

Also provided method of ameliorating at least one symptom of a genetic disease in a subject in need thereof, the method comprising administering at least one dose of a composition comprising a stabilized form of a protein for treating a genetic disorder, wherein the stabilized form comprising one or more non-native cysteine residues that form a disulfide bridge between non-native cysteines within the protein or between non-native cysteines of two monomers of the protein. In some embodiments, the modified polypeptide forms a homodimer. In some embodiments, the homodimer is stabilized by a disulfide bond. In some embodiments, the modified polypeptide shows increased half-life at pH 7.4 compared with a wild type polypeptide. In some embodiments, the at least one symptom is selected from one or more of mental impairment, seizures, loss of speech, and loss of motor skills. In some embodiments, the method further comprises administering a chaperone. In some embodiments, the chaperone comprises Migalastat.

In some embodiments, the composition is administered via intrathecal, intracerebroventricular, intraperenchymal, subcutaneous, intramuscular, ocular, intravenous injection, or a combination thereof

In some embodiments, the genetic disorder is a neurological disorder. In some embodiments, the genetic disorder is a lysosomal storage disorder. In some embodiments, genetic disorder is selected from the group consisting of aspartylglucosaminuria, Batten disease, cystinosis, Fabry disease, Gaucher disease type I, Gaucher disease type II, Gaucher disease type III, Pompe disease, Tay Sachs disease, Sandhoff disease, metachomatic leukodystrophy, mucolipidosis type I, mucolipidosis type II, mucolipidosis type III, mucolipidosis type IV, Hurler disease, Hunter disease, Sanfilippo disease type A, Sanfilippo disease type B, Sanfilippo disease type C, Sanfilippo disease type D, Morquio disease type A, Morquio disease type B, Maroteau-Lamy disease, Sly disease, Niemann-Pick disease type A, Niemann-Pick disease type B, Niemann-Pick disease type C1, Niemann-Pick disease type C2, Schindler disease type I, Schindler disease type II, adenosine deaminase severe combined immunodeficiency (ADA-SCID), chronic granulomatous disease (CGD), infantile, juvenile and adult forms of neuronal ceroid lipofuscinosis, and CDKL5 deficiency disease.

Also provided method of ameliorating at least one symptom of Fabry disease in a subject in need thereof, the method comprising administering at least one dose of a composition comprising a modified α-GAL polypeptide, wherein the modified α-GAL polypeptide comprises cysteine substitutions of an α-GAL polypeptide sequence. Contemplated cysteine substitutions include: (i) D233C and I359C; and (ii) M51C and G360C. In some embodiments, the modified α-GAL polypeptide cysteine substitutions of an α-GAL polypeptide sequence of D233C and I359C. In some embodiments, the modified α-GAL polypeptide cysteine substitutions of an α-GAL polypeptide sequence of M51C and G360C. In some embodiments, the modified α-GAL polypeptide forms a homodimer. In some embodiments, the homodimer is stabilized by a disulfide bond. In some embodiments, the modified α-GAL polypeptide shows increased half-life at pH 7.4 compared with a wild type α-GAL polypeptide. In some embodiments, the at least one symptom is selected from one or more of pain, skin discoloration, inability to sweat, eye cloudiness, gastrointestinal dysfunction, tinnitus, hearing loss, mitral valve prolapse, heart disease, joint pain, renal failure, and kidney dysfunction. In some embodiments, the method further comprises administering a chaperone. In some embodiments, the chaperone comprises Migalastat.

Also provided method of ameliorating at least one symptom of a CLN-1 disease in a subject in need thereof, the method comprising administering at least one dose of a composition comprising a modified PPT-1 polypeptide, wherein the PPT-1 polypeptide comprises one or more non-native cysteine residues that form a disulfide bridge between non-native cysteines within the protein or between non-native cysteines of two monomers of the protein. Contemplated cysteine substitutions include: A171C and A183C. In some embodiments, the modified PPT-1 polypeptide forms a homodimer. In some embodiments, the homodimer is stabilized by a disulfide bond. In some embodiments, the modified PPT-1 polypeptide shows increased half-life at pH 7.4 compared with a wild type PPT-1 polypeptide. In some embodiments, the at least one symptom is selected from one or more of mental impairment, seizures, loss of speech, and loss of motor skills. In some embodiments, the method further comprises administering a chaperone. In some embodiments, the chaperone comprises Migalastat.

In some embodiments, methods herein comprise administering modified polypeptides herein having an increased half-life compared with a wild type polypeptide. In some embodiments, the half-life is at least 50% greater than a wild type polypeptide. In some embodiments, the half-life is at least 150% greater than a wild type polypeptide. In some embodiments, the half-life is at least 200% greater than a wild type polypeptide. In some embodiments, the half-life is at least 250% greater than a wild type polypeptide. In some embodiments, the half-life is at least 300% greater than a wild type polypeptide. In some embodiments, the half-life is at least 350% greater than a wild type polypeptide.

In some embodiments, methods herein comprise administering modified α-GAL polypeptides herein having an increased half-life compared with a wild type α-GAL polypeptide. In some embodiments, the half-life is at least 50% greater than a wild type α-GAL polypeptide. In some embodiments, the half-life is at least 150% greater than a wild type α-GAL polypeptide. In some embodiments, the half-life is at least 200% greater than a wild type α-GAL polypeptide. In some embodiments, the half-life is at least 250% greater than a wild type α-GAL polypeptide. In some embodiments, the half-life is at least 300% greater than a wild type α-GAL polypeptide. In some embodiments, the half-life is at least 350% greater than a wild type α-GAL polypeptide.

In some embodiments, methods herein comprise administering modified PPT-1 polypeptides herein having an increased half-life compared with a wild type PPT-1 polypeptide. In some embodiments, the half-life is at least 50% greater than a wild type PPT-1 polypeptide. In some embodiments, the half-life is at least 150% greater than a wild type PPT-1 polypeptide. In some embodiments, the half-life is at least 200% greater than a wild type PPT-1 polypeptide. In some embodiments, the half-life is at least 250% greater than a wild type PPT-1 polypeptide. In some embodiments, the half-life is at least 300% greater than a wild type PPT-1 polypeptide. In some embodiments, the half-life is at least 350% greater than a wild type PPT-1 polypeptide.

### Definitions

Stabilized” as used herein with respect to a protein refers to a modified protein (e.g., modified to contain non-native cysteine residues) that maintains one or more of its biological activities for a period of time that is longer than a corresponding protein without the modification. In some embodiments, stabilized proteins maintain biological activity for a time that is about 10%, 20%, 30%, 40%, 50%, 60%, 70%, 80%, 90%, 100%, 150%, 200%, 250%, 300%, 350%, 400%, 450% or 500% longer than the corresponding protein without the modification. In some embodiments, stabilized proteins maintain biological activity for a time that is at least 10% longer, at least 20% longer, at least 30% longer, at least 40% longer, at least 50% longer, at least 60% longer, at least 70% longer, at least 80% longer, at least 90% longer, at least 100% longer, at least 150% longer, at least 200% longer, at least 250% longer, at least 300% longer %, at least 350% longer, at least 400% longer, at least 450% longer or at least 500% longer than the corresponding protein without the modification. In some embodiments, the stabilized protein has a longer half-life compared to a corresponding protein without the non-native cysteines. In some embodiments, the stabilized protein has a longer half-life at pH 4.0 to pH 8.0, or pH 4.0 to 6.0, or pH 6.0 to 8.0 compared to a corresponding protein without the non-native cysteines. In some embodiments, the stabilized protein has a longer half-life at pH 4.5 to 5.0 or 7.0 to 7.5 compared to a corresponding protein without the non-native cysteines. In some embodiments, the stabilized protein has a longer half-life at pH 7.4 compared to a corresponding protein without the non-native cysteines. In some embodiments, the stabilized protein has a longer half-life at pH 4.6 compared to a corresponding protein without the non-native cysteines.

As used herein “ex vivo gene therapy” refers to methods where patient cells are genetically modified outside the subject, for example to express a therapeutic gene. Cells with the new genetic information are then returned to the subject from whom they were derived.

As used herein “in vivo gene therapy” refers to methods where a vector carrying the therapeutic gene(s) is directly administered to the subject.

As used herein “fusion protein” and “therapeutic fusion protein” are used interchangeably herein and refer to a therapeutic protein having at least one additional protein, peptide, or polypeptide, linked to it. In some instances, fusion proteins are a single protein molecule containing two or more proteins or fragments thereof, covalently linked via peptide bond within their respective peptide chains, without chemical linkers. In some embodiments, the fusion protein comprises a therapeutic protein and a signal peptide, a peptide that increases endocytosis of the fusion protein, or both. In some embodiments, the peptide that increases endocytosis is a peptide that binds CI-MPR.

As used herein “plasmid” refers to circular, double-stranded unit of DNA that replicates within a cell independently of the chromosomal DNA.

As used herein “promoter” refers to a site on DNA to which the enzyme RNA polymerase binds and initiates the transcription of DNA into RNA.

As used herein “somatic therapy” refers to methods where the manipulation of gene expression in cells that will be corrective to the patient but not inherited by the next generation. Somatic cells include all the non-reproductive cells in the human body

As used herein “somatic cells” refers to all body cells except the reproductive cells.

As used herein “tropism” refers to preference of a vector, such as a virus for a certain cell or tissue type. Various factors determine the ability of a vector to infect a particular cell. Viruses, for example, must bind to specific cell surface receptors to enter a cell. Viruses are typically unable to infect a cell if it does not express the necessary receptors.

As used herein “vector”, or “gene therapy vector”, used interchangeably herein, refers to gene therapy delivery vehicles, or carriers, that deliver therapeutic genes to cells. A gene therapy vector is any vector suitable for use in gene therapy, e.g., any vector suitable for the therapeutic delivery of nucleic acid polymers (encoding a polypeptide or a variant thereof) into target cells (e.g., sensory neurons) of a patient. In some embodiments, the gene therapy vector delivers the nucleic acid encoding a therapeutic protein or therapeutic fusion protein to a cell where the therapeutic protein or fusion is expressed and secreted from the cell. The vector may be of any type, for example it may be a plasmid vector or a minicircle DNA. Typically, the vector is a viral vector. These include both genetically disabled viruses such as adenovirus and nonviral vectors such as liposomes. The viral vector may for example be derived from an adeno-associated virus (AAV), a retrovirus, a lentivirus, a herpes simplex virus, or an adenovirus. AAV derived vectors. The vector may comprise an AAV genome or a derivative thereof.

“Construct” as used herein refers to a nucleic acid molecule or sequence that encodes a therapeutic protein or fusion protein and optionally comprises additional sequences such as a translation initiation sequence or IRES sequence.

The term “transduction” is used to refer to the administration/delivery of the nucleic acid encoding the therapeutic protein to a target cell either in vivo or in vitro, via a replication-deficient rAAV of the disclosure resulting in expression of a functional polypeptide by the recipient cell. Transduction of cells with a gene therapy vector such as a rAAV of the disclosure results in sustained expression of polypeptide or RNA encoded by the rAAV. The present disclosure thus provides methods of administering/delivering to a subject a gene therapy vector such as an rAAV encoding a therapeutic protein by an intrathecal, intraretinal, intraocular, intravitreous, intracerebroventricular, intraparenchymal, or intravenous route, or any combination thereof. “Intrathecal” delivery refers to delivery into the space under the arachnoid membrane of the brain or spinal cord. In some embodiments, intrathecal administration is via intracisternal administration.

The terms “recipient”, “individual”, “subject”, “host”, and “patient”, are used interchangeably herein and in some cases, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human.

As used herein, the terms “treatment,” “treating,” “ameliorating a symptom,” and the like, in some cases, refer to administering an agent, or carrying out a procedure, for the purposes of obtaining a therapeutic effect, including inhibiting, attenuating, reducing, preventing or altering at least one aspect or marker of a disorder, in a statistically significant manner or in a clinically significant manner. The term “ameliorate” or “treat” does not state or imply a cure for the underlying condition. “Treatment,” or “to ameliorate” (and like) as used herein, may include treating a mammal, particularly in a human, and includes: (a) preventing the disorder or a symptom of a disorder from occurring in a subject which may be predisposed to the disorder but has not yet been diagnosed as having it (e.g., including disorders that may be associated with or caused by a primary disorder; (b) inhibiting the disorder, i.e., arresting its development; (c) relieving the disorder, i.e., causing regression of the disorder; and (d) improving at least one symptom of the disorder. Treating may refer to any indicia of success in the treatment or amelioration or prevention of a disorder, including any objective or subjective parameter such as abatement; remission; diminishing of symptoms or making the disorder condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with the disorder. The term “therapeutic effect” refers to the reduction, elimination, or prevention of the disorder, symptoms of the disorder, or side effects of the disorder in the subject.

The term “affinity” refers to the strength of binding between a molecule and its binding partner or receptor.

As used herein, the phrase “high affinity” refers to, for example, a therapeutic fusion containing such a peptide that binds CI-MPR which has an affinity to CI-MPR that is about 100 to 1,000 times or 500 to 1,000 times higher than that of the therapeutic protein without the peptide. In some embodiments, the affinity is at least 100, at least 500, or at least 1000 times higher than without the peptide. For example, where the therapeutic protein and CI-MPR are combined in relatively equal concentration, the peptide of high affinity will bind to the available CI-MPR so as to shift the equilibrium toward high concentration of the resulting complex.

“Secretion” as used herein refers to the release of a protein from a cell into, for example, the bloodstream to be carried to a tissue of interest or a site of action of the therapeutic protein. When a gene therapy product is secreted into the interstitial space of an organ, secretion can allow for cross-correction of neighboring cells.

“Delivery” as used herein means drug delivery. In some embodiments, the process of delivery means transporting a drug substance (e.g., therapeutic protein or fusion protein produced from a gene therapy vector) from outside of a cell (e.g., blood, tissue, or interstitial space) into a target cell for therapeutic activity of the drug substance.

“Engineering” or “protein engineering” as used here in refers to the manipulation of the structures of a protein by providing appropriate a nucleic acid sequence that encodes for the protein as to produce desired properties, or the synthesis of the protein with particular structures.

A “therapeutically effective amount” in some cases means the amount that, when administered to a subject for treating a disease, is sufficient to effect treatment for that disease.

As used herein, the term “about” a number refers to a range spanning that from 10% less than that number through 10% more than that number, and including values within the range such as the number itself.

As used herein, the term “comprising” an element or elements of a claim refers to those elements but does not preclude the inclusion of an additional element or elements.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Identifying Amino Acid Residues for Cysteine Substitution of Wild Type α-GAL

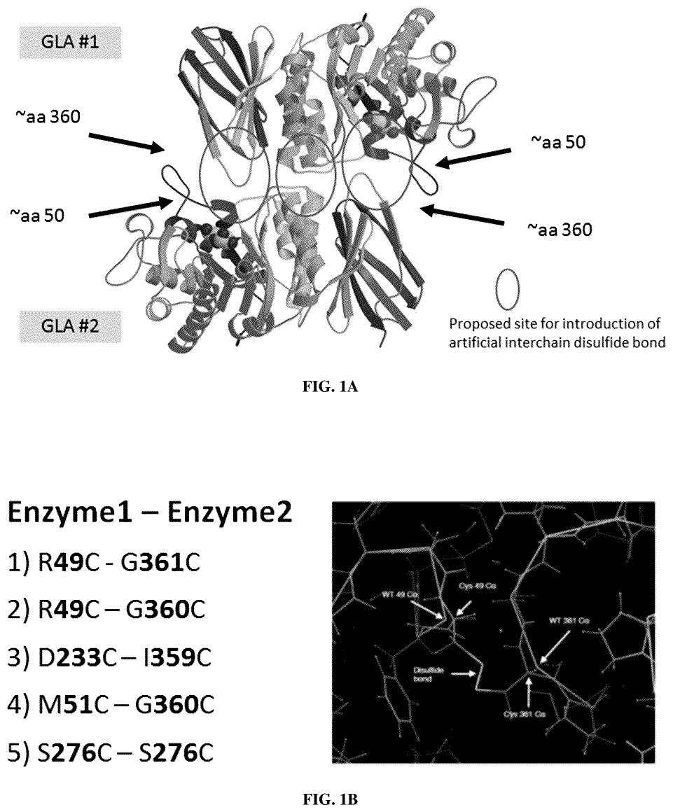

The crystal structure of dimerized α-GAL (PDB ID 3HG3) was examined for potential sites for substituting in cysteine residues, generating additional disulfide bonds for enhanced stability (FIG. 1A). NAMD with CHARMM forcefields was used for the analysis. Based on the analysis, the cysteine mutants shown in Table 8 were prepared using standard methods of directed mutagenesis.

TABLE 8
α-GAL Disulfide Mutants

| | SEQ |
|---|---|
| Mutations | ID NO |
| R49C-G361C | 2 |
| R49C-G360C | 3 |
| M51C-G360C | 4 |
| D233C-I359C | 5 |
| S276C | 6 |

See also FIG. 1B. Amino acid sequences are provided in Table 1.

### Example 2: Dimerization and Enzymatic Activity of Modified α-GAL

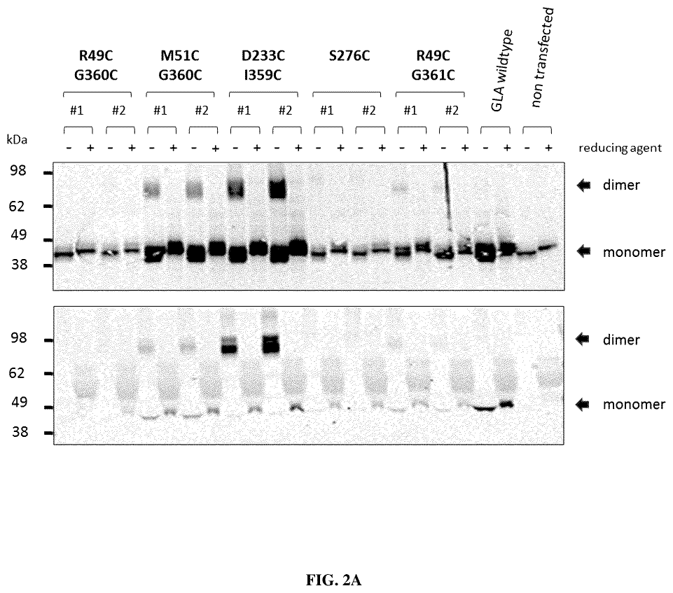

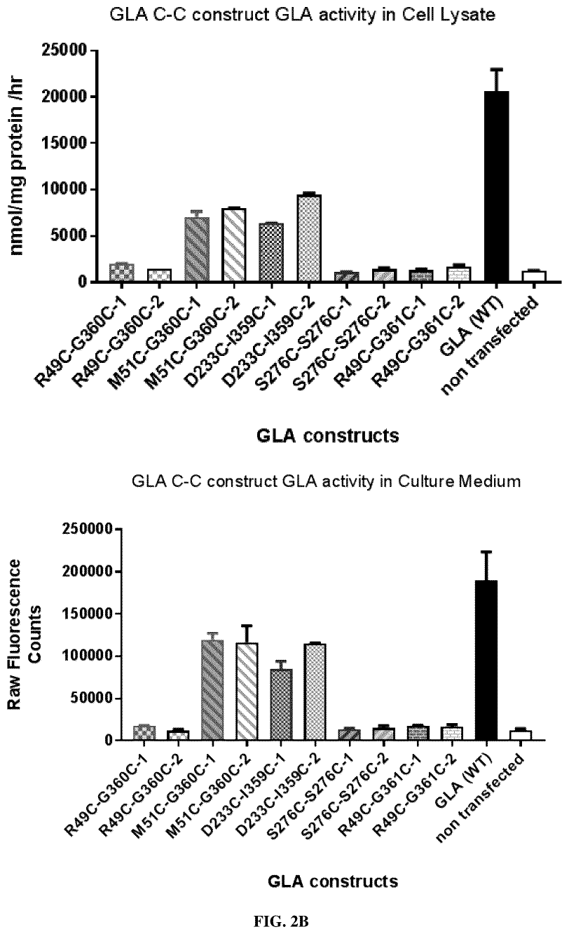

The formation of disulfide bonded dimers of modified α-GAL was examined in cell lysate and culture media (FIG. 2A). Clones of each α-GAL construct were transiently expressed in 293HEK cell. Cell lysates and culture media were run on 4-12% gradient SDS-PAGE and transferred to nitrocellulose. α-GAL was detected by Western Blotting with rabbit monoclonal anti-α-GAL 1:2000 (abcam ab168341).

Reduced and non-reduced samples were subjected to electrophoresis and Western blotting. As seen in FIG. 2, M51C-G360C and D233C-I359C versions of the α-GAL readily formed disulfide bonded α-GAL dimers.

To prepare the samples, 1×10\hat{ }6 cells were harvested with transient expression of α-GAL constructs. Cells were lysed in 500 ul 20 mM sodium phosphate buffer pH6.5, 0.25% TX-100. Cell lysate was centrifuged for 2 min @10,000 g and transfer supernatant to new tube. 40 ul of cell lysate or culture media was transferred to new tube and 16 μl of LDS 4× Sample Buffer was added with 6 μl of 10× Reducing agent (for reducing conditions). Sample mix prepared as below was heated at 95° C. for 5 minutes. 1×MOPS SDS running buffer was used for electrophoresis.

To test for enzymatic activity, lysate or culture medium were incubated with 4-methylumbelliferone-α-D-galactopyranoside (4-MUG) substrate for 1 hour. Enzymatic reaction was then stopped, and the α-GAL enzymatic activity was measured by fluorescence at excitation 360 nm and emission at 450 nm. As shown in FIG. 2B, the M51C-G360C and D233C-I359C disulfide α-GAL mutants were both enzymatically active. Because the specific activity and amount of α-GAL in each sample were not quantified, FIG. 2B does not provide a quantitative comparison of the activity between the wild type and mutant versions of α-GAL.

### Example 3: Stability Analysis of Modified α-GAL in Acidic Environments Over Time

To test pH stability over 24 h, transiently expressed mutant and wildtype α-GAL was captured using Concanavalin A (ConA) agarose pull-down according to standard methods. The ConA eluate was diluted in either pH 4.6 buffer or pH 7.4 buffer. Samples were pre-incubated at pH 4.6 or 7.4 at 0, 0.5, 1, 2, 4, 5 and 24 hours.

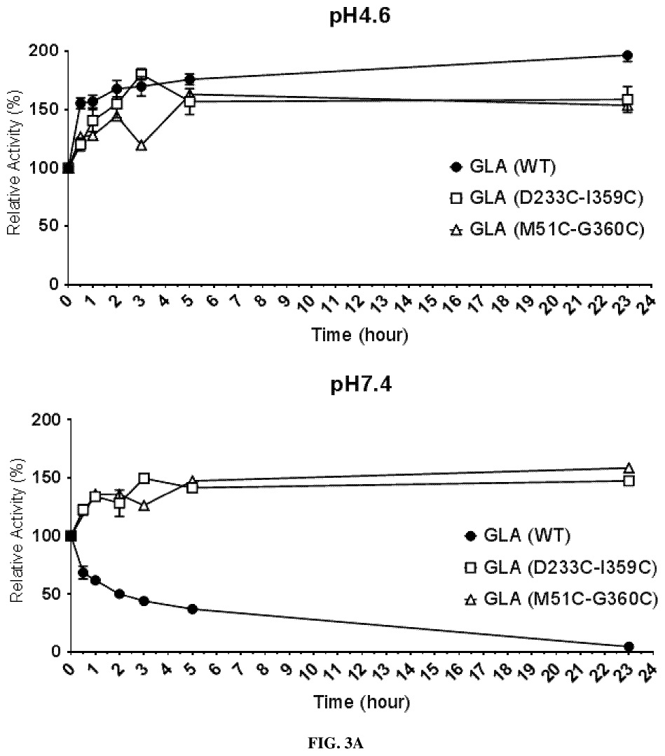

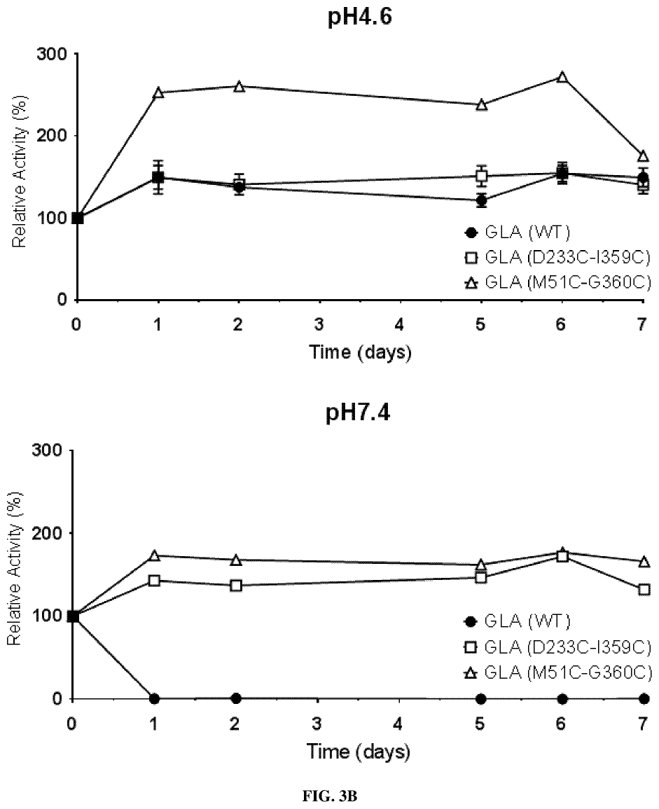

To measure enzyme activity pH 4.6 buffer was added to each sample and tested for activity on a 4-MUG substrate. The reaction mixture was incubated 37° C. for 1 hour. The reaction was terminated by adding 125 uL Stop buffer (0.4 M Glycin-NaOH, pH 10.8) Fluorescence was read with Spectramax plate reader: Ex: 360 nm, Em: 450 nm. The results are shown in FIG. 3A.

For long-term stability testing, transiently expressed modified and wild type α-GALs were isolated from culture media and enriched and purified using ConA agarose beads as described above. The eluted α-GAL was incubated in pH 4.6 or pH 7.4 for time course stability experiments. The time points included 0 hr, 0.5 hr, 1 hr, 2 hr, 4 hr, 5 hr, 24 hr, 2 days, 5 days, 6 days, and 7 days. FIG. 3B shows M51C-G360C α-GAL to be more stable over a span of 7 days than the wild type control at pH 4.6. Both modified α-GALs were substantially more stable than wild type control at pH 7.4 over 7 days.

### Example 4: α-GAL Uptake and Enzymatic Assay by Fabry Patient Fibroblasts

### Cell Uptake Protocol

To conduct the uptake assay, on day 1 300,000 Fabry patient fibroblasts (R301Q) were seeded per well in 6-well plates. On day 2, medium was replaced with 1.8 mL uptake medium and incubated for 1 hour at 37 C with 5% CO2. Cells were given a 200 uL dose of 250 nM enzyme (Fabrazyme, M51C-G360C, D233C-I359C and WT) prepared in uptake medium into 6-well prepared in step 2 for 16-18 hours. On day 3, 300 μL of 1 M Tris was added and incubated at room temperature for 30 min. 400 uL 1M NaH2SO4 was added and mixed. Cell plates were washed with 1 ml DPBS two times. 500 uL water was added into each well and cells were collected from the plate. Matric-green was added before freezing at −80° C. freezer until assay. Plates were spun before enzyme and protein assays.

The protein assay was conducted by adding 20 uL cell lysate into 130 uL water. 150 uL BCA working reagent was added and incubated at 37 C.° for 2 hours. The plate was then read on a Spectramax.

The enzyme assay was conducted by adding 5 uL cell lysate into 15 uL Assay Buffer then adding 50 uL 4-MUG substrate. This was incubated at 37° C. for 1 hour. 125 uL Stop Buffer was added and read at the Spectramax.

As a control, frozen cell lysates were thawed at room temperature and sonicated for 5 min. 50 μL was transferred into 13 mL silanized glass tubes. 25 μL of Glucopsychosine (IS) (conc. 125 ng/mL) was added. 1 mL of methanol was added and the mixture was sonicated for approximately 10 min. 500 μL of 1N HCl was added, vortexed then sonicated for approximately 10 minutes. The mixture was then shaken for approximately 30 minutes at room temperature. Samples were centrifuged at 4,000 rpm for 10 min. at room temperature. Supernatant was transferred onto preconditioned SPE cartridges.

Solid samples were prepared by condition the SPE cartridges with 1 ml of methanol and 1 ml of Millipore water. Samples were loaded on the SPE cartridges. Cartridges were washed with 2 mL 0.1N HCl and then 2 mL MEOH. Samples were eluted with 2 mL 5% ammonium hydroxide in methanol into clean silanized glass. Samples were evaporated under nitrogen to dryness at 40° C. 25 μL of DMSO was added to each extract and vortexed. 125 μL (175 μL was used for run 03) of mobile phase B was added and vortexed. Samples were transferred into glass vials. 10 μl was injected onto analytical column.

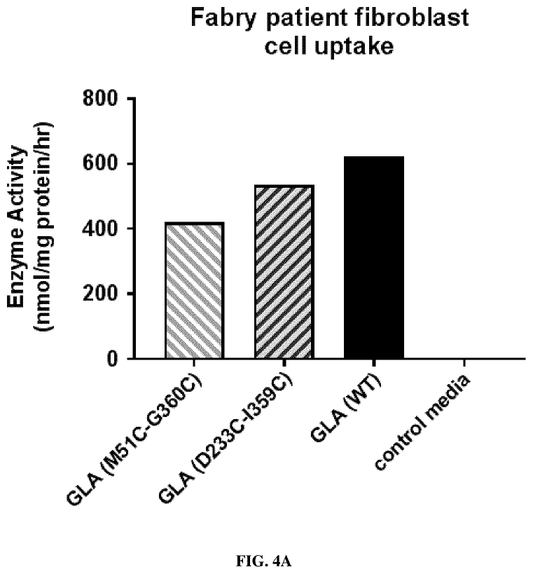

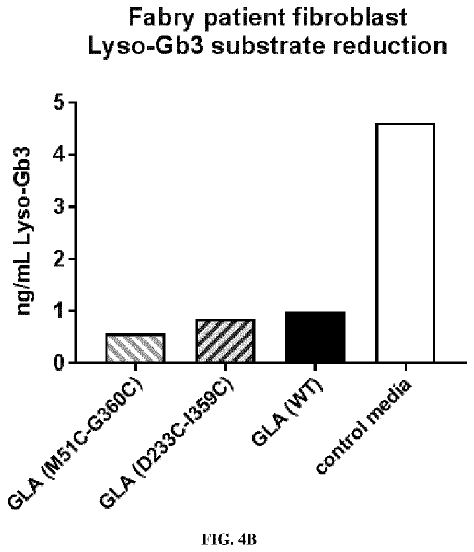

Fibroblasts from Fabry disease patients were cultured and seeded in 6-well plates. Cells treated with wild type α-GALs were used as positive control for the uptake and subsequent enzymatic studies. Fibroblasts were incubated for 16 to 18 hr with wild type α-GAL, M51C-G360C α-GAL, or D233C-I359C α-GAL. The cells were then lysed for α-GAL enzymatic assay as determined by fluorescent output. FIG. 4A shows that both M51C-G360C and D233C-I359C α-GALs were able to restore α-GAL enzymatic activity at least as well wild type α-GAL. See FIG. 4B. Globotriaosylsphingosine (lyso-Gb3) is a biomarker for Fabry Disease. Successful treatment of Fabry Disease leads to significant reduction of lyso-Gb3 as determined by LC-MS/MS.

### Example 5: Variant Homodimers Uptake in FB-14 (R301Q) Fabry Patient Fibroblasts

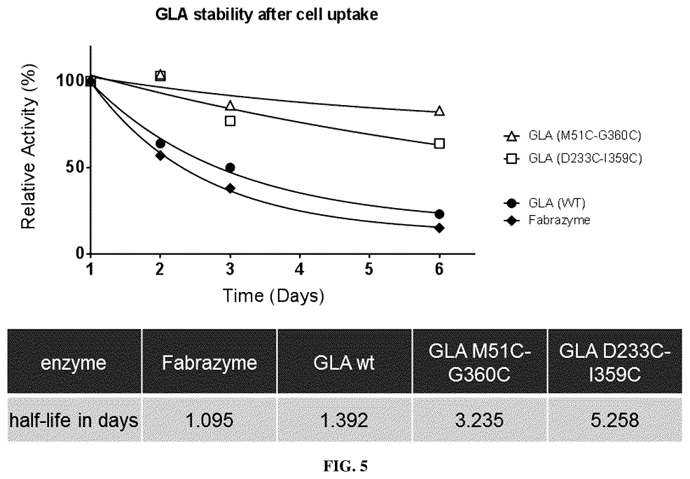

Fabry patient fibroblast cells were seeded in 6-well plate for Fabrazyme and α-GAL cell uptake studies. Cells were incubated for 16 h at 37 C, 5% CO2 incubator in uptake media containing 7 nM of either Fabrazyme, wild type α-GAL, M51C-G360C α-GAL, or D233C-I359C α-GAL. At day 1 cells were washed and further maintained in regular growth media for 5 additional days. Cells were harvested at time points indicated in FIG. 5. Cell lysates were used to determine enzyme activity. α-GAL enzyme activity was determined and normalized with cell lysate protein concentration as nmol/mg protein/hr. It was determined that the variant homodimers have 2-3-fold longer half-life inside the cell after cell uptake than wildtype and 3-4-fold longer than Fabrazyme (FIG. 5).

### Example 6: Fabry Disease Gene Therapy in Mouse Model

The AAV vectors were diluted in sterile PBS. The AAV vectors included: AAVhu68.CB7.hGLAnatural.rBG, AAVhu68.CB7.hGLAco.rBG, and AAVhu68.CB7.hGLA-M51C-G360Cco.rBG.

### Vector Production

The reference GLA sequence and the variant with the methionine to cysteine at position 51 and glycine to cysteine at position 360 were back-translated and the nucleotide sequence was codon optimized to generate a cis-plasmid for AAV production with the expression cassette under CB7 promoter. In addition, natural hGLA (reference sequence) cDNA was ordered and cloned into the same AAV-cis backbone to compare with a codon-optimized sequence. AAVhu68 vectors were produced and titrated as previously described (Lock, Alvira et al. 2010, “Rapid, simple, and versatile manufacturing of recombinant adeno-associated viral vectors at scale.” Hum Gene Ther 21(10): 1259-1271). Briefly, HEK293 cells were triple-transfected and the culture supernatant was harvested, concentrated, and purified with an iodixanol gradient. The purified vectors were titrated with droplet digital PCR using primers targeting the rabbit Beta-globin polyA sequence as previously described (Lock M, R. Alvira, S. J. Chen and J. M. Wilson, “Absolute determination of single-stranded and self-complementary adeno-associated viral vector genome titers by droplet digital PCR.” Hum Gene Ther Methods 25(2): 115-125 (2014)).

### Animals

Mus musculus, Fabry mice Gla knock-out, in a C57BL/6/129 background founders were purchased at Jackson Labs (stock #003535—“also known as” α-Gal A KO mice”). The breeding colony was maintained at the Gene Therapy Program AAALAC accredited barrier mouse facility, using heterozygote to heterozygote mating in order to produce null and WT controls within the same litters. The Gla knock-out mouse is a widely used model for Fabry disease.

The mice appear clinically normal, but they exhibit a progressive accumulation of the GLA substrate Globotriaosylsphingosine (aka lyso-GB3) in plasma and Globotriaosylceramide (aka GL3, GB3) in liver, heart, kidney, skin small and large intestine and the central nervous system. The small size, reproducible phenotype, and efficient breeding allow quick studies that are optimal for preclinical candidates in vivo screening.

Animal holding rooms were maintained at a temperature range of 64-79° F. (18-26° C.) with a humidity range of 30-70%. Animals were housed with their parents and littermates until weaning and next in standard caging of 2 to 5 animals per cage in the Translational Research Laboratories (TRL) GTP vivarium. Cages, water bottles, and bedding substrates are autoclaved into the barrier facility. An automatically controlled 12-hour light/dark cycle was maintained. Each dark period began at 1900 hours (±30 minutes). Food was provided ad libitum (Purina, LabDiet®, 5053, Irradiated, PicoLab®, Rodent Diet 20, 251b). Water was accessible to all animals ad libitum via individually placed water bottle in each housing cage.

### In Vivo Studies and Histology

Mice received 5×10¹¹ GCs (approximately 2.5×10³ GC/kg) of AAVhu68.CB7.hGLA (various hGLA constructs) in 0.1 mL via the lateral tail vein, were bled on Day 7 and Day 21 post vector dosing for serum isolation and were terminally bled (for plasma isolation) and euthanized by exsanguination 28 days post injection. Tissues were promptly collected, starting with brain.

Tissues for histology were formalin-fixed and paraffin embedded using standard methods. Spinal cord with DRG (in bone) was fixed in ZF, decalcified in EDTA and processed according to standard procedures of the GTP Morphology Core. Zinc-formalin is used to obtain good tissue preservation and was used to stain the Gb3 storage by IHC and for morphology (H&E).

Immunostaining for GL3 was performed on formalin-fixed paraffin-embedded samples. Sections were deparaffinized, blocked with 1% donkey serum in PBS+0.2% Triton for 15 min, and then sequentially incubated with primary (Amsbio AMS.A2506, anti-Gb3 monoclonal antibody) and biotinylated secondary antibodies diluted in blocking buffer; an HRP based colorimetric reaction was used to detect the signal. Slides were reviewed in a blinded fashion by a board-certified Veterinary Pathologist.

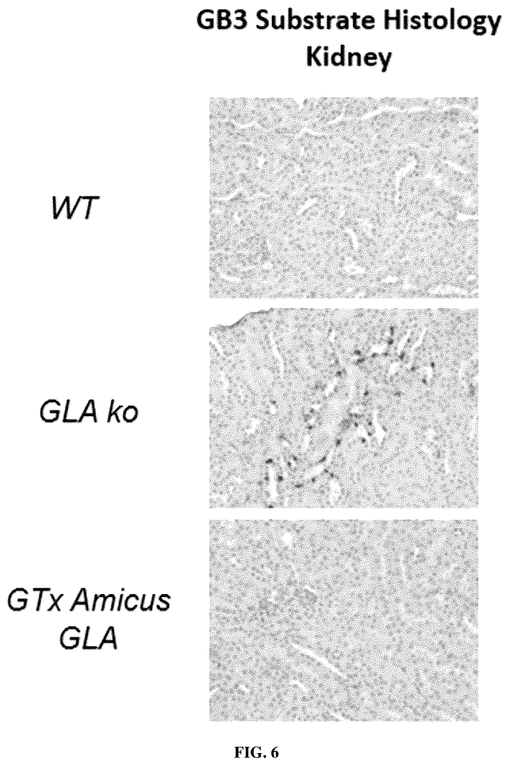

Fabry −/− mice vehicle PBS controls display marked GL3 (dark staining on IHC stained sections) accumulation. WT mice and all vector treated mice have near complete to complete clearance of GL3 storage (FIG. 6).

### GLA Activity

Plasma or supernatant of homogenized tissues were mixed with 6 mM 4-MU-α-galactopyranoside pH 4.6, 90 mM GalNAc and incubated for three hours at 37° C. The reaction was stopped with 0.4 M glycine pH 10.8. Relative fluorescence units, RFUs were measured using a Victor3 fluorimeter, ex 355 nm and emission at 460 nm. Activity in units of nmol/mL/hr was calculated by interpolation from a standard curve of 4-MU. Activity levels in individual tissue samples were normalized for total protein content in the homogenate supernatant. Equal volumes are used for plasma samples.

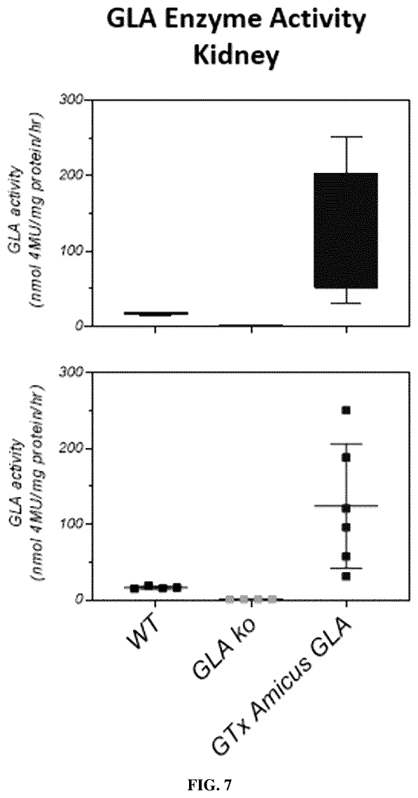

Fabry −/− mice displayed a complete lack of α-Gal A activity. Treatment of Fabry mice with AAVhu68.CB7.hGLA-M51C-G360Cco.rBG GTx vector resulted in >7-fold higher GLA activity in kidney than wildtype (FIG. 7).

### Quantitation of Globotriaosylceramide (Aka GL3, GB3) by LC-MS/MS

The GLA substrate, GL3, in tissue homogenate was quantified by a LC-MS/MS assay. Briefly, an internal standard was added to homogenate samples (50 μL) and the samples were processed using C18-based solid-phase extraction (SPE). A standard curve was prepared to known concentrations of GL3 (8.83 nM to 4.41 μM) from stocks containing twelve ceramide forms. Monitored responses from all twelve isoforms were to be summed and a ratio was generated with respect to internal standard in this assay. The resultant ratios of study samples were then compared against the prepared curve for GL3 quantification.

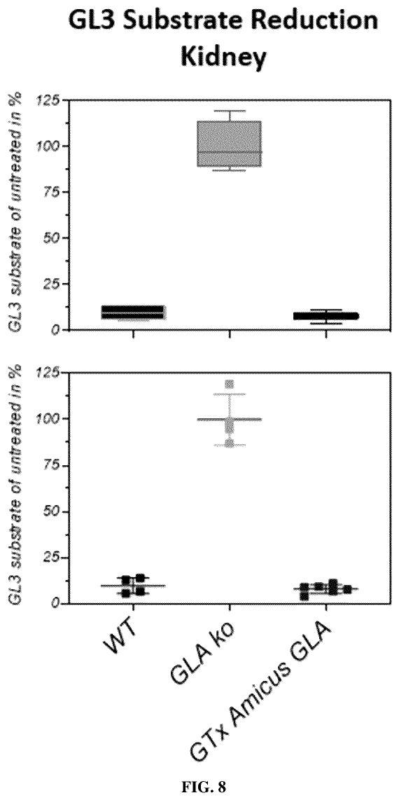

Fabry −/− mice displayed a >10-fold accumulation of the GLA substrate Globotriaosylceramide (GL3). Treatment of Fabry mice with AAVhu68.CB7.hGLA-M51C-G360Cco.rBG GTx vector resulted in a completed reduction of GL3 in kidney to wildtype level (FIG. 8).

### Quantitation of Globotriaosylsphingosine (Aka Lyso-GB3) by LC-MS/MS

The GLA substrate, lyso-GB3, in plasma is quantified by a LC-MS/MS assay. Briefly, a stable C13-labeled internal standard is added to the plasma samples (50 μL) and the samples are processed using C18/cation exchange mixed mode solid-phase extraction (SPE). A standard curve is prepared to known concentrations of lyso-GB3 (0.254 nM to 254 nM) and lyso-GB3 response of study samples are then compared against the prepared curve for lyso-GB3 quantification.

### GLA Signature Peptide by LC/MS

Plasma is precipitated in 100% methanol and centrifuged. Supernatants are discarded. The pellet is spiked with a stable isotope-labeled peptide unique to hGLA as an internal standard and resuspended with trypsin and incubated at 37° C. for two hours. The digestion is stopped with 10% formic acid. Peptides are separated by C-18 reverse phase chromatography and identified and quantified by ESI-mass spectroscopy. The total GLA concentration in plasma is calculated from the signature peptide concentration.

### Cell Surface Receptor Binding Assay

A 96-well plate is coated with receptor, washed, and blocked with BSA. CHO culture conditioned media or plasma containing equal activities of either rhGLA or engineered GLA is serially diluted three-fold to give a series of nine decreasing concentrations and incubated with co-coupled receptor. After incubation the plate is washed to remove any unbound GLA and 4-MU-α-galactopyranoside added for one hour at 37° C. The reaction is stopped with 1.0 M glycine, pH 10.5 and RFUs were read by a Spectramax fluorimeter; ex 370, emission 460. RFU's for each sample and are converted to activity in nmol/mL/hr by interpolation from a standard curve of 4-MU. Nonlinear regression is done using GraphPad Prism.

### Example 7: Stabilized PPT-1 Constructs

A stabilized PPT-1 construct was engineered based on the crystal structure (PDB ID 3GRO). These two cysteines are predicted to form a disulfide bond, which stabilizes the structure and was found to extend the half-life of enzymatic function (see data below). The expression level of this construct in HEG 293 was found to be close to that of wildtype PPT-1.

### Improved Stability and Half-Life of PPT-1 Enzyme

An intramolecular disulfide bridge was engineered into PPT-1 in order to stabilize the enzyme, as determined by measuring the half-life of the active enzyme. The residues that were mutated, A171C/A183C, were chosen because the equivalent residues in a homologous protein, PPT-2, form a disulfide bridge. This natural variation in a homologous protein was used to inform the engineering efforts in PPT-1.

### Stability Testing of Construct PPT-1

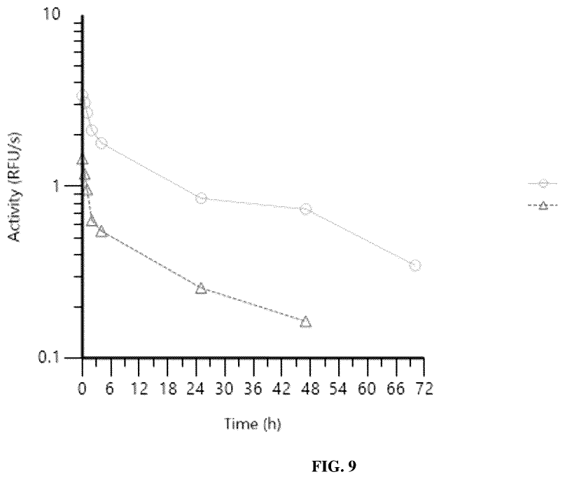

Construct PPT-1 was expressed transiently in HEK 293T cells and the conditioned media was harvested five days post-transfection. The same was done for wildtype PPT1. Enzyme activity assays were performed on both sets of conditioned media over a course of 48 or 72 hours. The amount of enzymatic activity retained over time was determined in order to compare the cysteine double mutant with WT. (FIG. 9).

Half-life was estimated in two ways representing the alpha and beta phases, as activity appears to be a biphasic elimination (see log plot adjacent to the PK table below). The alpha half-life was estimated during the early terminal or distribution phase, the beta half-life was estimated during the terminal elimination phase. For ATB200 total GAA protein analyses, the alpha phase is often reported as it is more meaningful for demonstrating effect of AT2221 on binding and stabilization of ATB200 while in blood, during distribution into tissues.

Pharmacokinetics of Construct PPT-1, including C₀ and AUCs, are reported. The AUC_{infinity} was derived from the same elimination rate constant used to estimate the beta half-life.

TABLE 9
PPT-1 Pharmacokinetics (5 day average)

| Construct | C₀ | AUC₀₋ₜ | AUC_{0-∞} | t_{1/2α} | t_{1/2β} |
|---|---|---|---|---|---|
| Wildtype | 1.46 | 15.9 | 21.0 | 1.7 | 23.1 |
| Cys-mutant | 3.40 | 65.6 | 80.3 | 2.8 | 28.2 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A gene therapy vector comprising a nucleic acid construct comprising: a nucleic acid encoding a stabilized form of a protein for treating a genetic disorder, the stabilized form comprising one or more non-native cysteine residues that form a disulfide bridge between non-native cysteines within the protein or between non-native cysteines of two monomers of the protein.

2. A method for treating a genetic disorder in a subject comprising administering to the subject a therapeutically effective amount of the gene therapy vector of claim 1.


SEQUENCE LISTING (34 sequences)

SEQ ID NO: 1 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 2 - protein, 429 aa, synthetic construct
  REGION 1..429: note = source = /note="Description of Artificial Sequence: Synthetic polypeptide"
MQLRNPELHLGCALALRFLALVSWDIPGARALDNGLARTPTMGWLHWECFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGCPRSYTIAVASLGKGVACNPACFITQLLPVKRKLGFYEWTSRLRSHINPTGTVLLQLENTMQMSLKDLL

SEQ ID NO: 3 - protein, 429 aa, synthetic construct
  REGION 1..429: note = source = /note="Description of Artificial Sequence: Synthetic polypeptide"
MQLRNPELHLGCALALRFLALVSWDIPGARALDNGLARTPTMGWLHWECFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEICGPRSYTIAVASLGKGVACNPACFITQLLPVKRKLGFYEWTSRLRSHINPTGTVLLQLENTMQMSLKDLL

SEQ ID NO: 4 - protein, 429 aa, synthetic construct
  REGION 1..429: note = source = /note="Description of Artificial Sequence: Synthetic polypeptide"
MQLRNPELHLGCALALRFLALVSWDIPGARALDNGLARTPTMGWLHWERFCCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEICGPRSYTIAVASLGKGVACNPACFITQLLPVKRKLGFYEWTSRLRSHINPTGTVLLQLENTMQMSLKDLL

SEQ ID NO: 5 - protein, 429 aa, synthetic construct
  REGION 1..429: note = source = /note="Description of Artificial Sequence: Synthetic polypeptide"
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADICDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQECGGPRSYTIAVASLGKGVACNPACFITQLLPVKRKLGFYEWTSRLRSHINPTGTVLLQLENTMQMSLKDLL

SEQ ID NO: 6 - protein, 429 aa, synthetic construct
  REGION 1..429: note = source = /note="Description of Artificial Sequence: Synthetic polypeptide"
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLCWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 7 - DNA, 1290 bp, Homo sapiens
  source 1..1290: mol_type = other DNA
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 8 - DNA, 1290 bp, synthetic construct
  misc_feature 1..1290: note = source = /note="Description of Artificial Sequence: Synthetic polynucleotide"
  source 1..1290: mol_type = other DNA
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagtgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggttgccctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 9 - DNA, 1290 bp, synthetic construct
  misc_feature 1..1290: note = source = /note="Description of Artificial Sequence: Synthetic polynucleotide"
  source 1..1290: mol_type = other DNA
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagtgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagatttgtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 10 - DNA, 1290 bp, synthetic construct
  misc_feature 1..1290: note = source = /note="Description of Artificial Sequence: Synthetic polynucleotide"
  source 1..1290: mol_type = other DNA
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttctgctgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagatttgtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 11 - DNA, 1290 bp, synthetic construct
  misc_feature 1..1290: note = source = /note="Description of Artificial Sequence: Synthetic polynucleotide"
  source 1..1290: mol_type = other DNA
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacatttgcgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagtgcggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 12 - DNA, 1290 bp, synthetic construct
  misc_feature 1..1290: note = source = /note="Description of Artificial Sequence: Synthetic polynucleotide"
  source 1..1290: mol_type = other DNA
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctctgctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 13 - protein, 306 aa, Homo sapiens
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 14 - protein, 306 aa, synthetic construct
  REGION 1..306: note = source = /note="Description of Artificial Sequence: Synthetic polypeptide"
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGCYSKVVQERLVQCEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 15 - DNA, 2504 bp, Homo sapiens
  source 1..2504: mol_type = other DNA
ttattttgattcaccgcagagggcggtctacgagagcgcagagccccactcggccagcggggtctggcgggggacctgtcgcgctgaaagctccagggtagggccgacgcccatcaggctgggcatccgttcgggatgcgcaggttgcgatctgcaaccggcggcgccacgcccaggcgggcggagcgcggttcccggagtctcgcgcccgcggtcatgtgacacagcgaagatggcgtcgcccggctgcctgtggctcttggctgtggctctcctgccatggacctgcgcttctcgggcgctgcagcatctggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggatgaaacccgtatagttcacaatagagctcagggagcccctaactcttccaaaccacatgggagacagtttccttcatgcccaagcctgagctcagatccagcttgcaactaatccttctatcatctaacatgccctacttggaaagatctaagatctgaatcttatcctttgccatcttctgttaccatatggtgttgaatgcaagtttaattaccatggagattgttttacaaacttttgatgtggtcaagttcagttttagaaaagggagtctgttccagatcagtgccagaactgtgcccaggcccaaaggagacaactaactaaagtagtgagatagattctaagggcaaacatttttccaagtcttgccatatttcaagcaaagaggtgcccaggcctgaggtactcacataaatgctttgttttgctggtgatttaaccagtgcttggaaaaatcttgcttggctatttctgcatcatttcttaaggctgccttcctctctcagtacgttgccctctgtgctatcatcttatcatcaattattagacaaatcccactggcctacagtcttgcttctgcagcacccactttgtctcctcaggtagtgatgaattagttgctgtcacaaaaggagggaagtagcacccaaattaagttgcttaagagaggaaatgtacatcttgtataacttagggagcgaagaaaatgtaggcgcgaaagtgaaaagtgaggcagctagttcttcctattccattctcgaccaacctgccctttcttaatatgactagtggtcttgatgctagagtcaacttactctgttgctggctttagcagagaataggaggaaccatatgaaaaagatcaggctttctgacttccatccccaaaacacatttaccagcatactccaaactgtttctgatgtgttccatgagaaaaggattgtttgctcaaaaagcttggaaaatactacacactccctttctccttctggagatcaacccacattagagtgtctaaggactcctgagaattcctgttacagtaaacaaaactaacgtaatctaccatttcctacactatttgagcatggaaatcatagtccccactctgtgaaaacttaacgctttttggaagacatttctgtagcatgtcagtttggagaaatgatgagctacgccttgatgaaagaaccgtgttggtgctgctaagtttagccattatggtttttcctttctctctcttaagccttattcttcaactaaaagatgaggattaagagcaagaagttgggggggatgtgaaaataattttatgaggttgtctaaaataaagagtagtttcttatc

SEQ ID NO: 16 - DNA, 921 bp, synthetic construct
  misc_feature 1..921: note = source = /note="Description of Artificial Sequence: Synthetic polynucleotide"
  source 1..921: mol_type = other DNA
atggcatcaccgggttgcctctggttgttggccgttgcgttgcttccgtggacatgtgcatcaagagctcttcaacatctggatcccccagctcccctgccgctcgtaatctggcacgggatgggggattcatgttgtaacccgttgtcaatgggcgcgataaaaaagatggttgaaaagaagattccaggcatctacgttctgtccctggaaatcggtaagacactgatggaagacgtggagaactccttctttctcaacgtcaatagtcaggtcactaccgtctgtcaagcattggcaaaggaccctaaacttcagcaggggtacaatgcgatggggtttagccagggcggacagtttcttagagccgtcgcacagcgctgtccatctcccccgatgattaaccttatatctgtcgggggacaacaccagggtgtttttggtcttcctcgctgtcctggtgaaagctcccacatctgtgatttcatacgcaaaacgttgaacgcaggatgctatagtaaagtcgtccaagaacggcttgttcaatgcgagtattggcatgacccaataaaagaagacgtttataggaatcactctatcttcttggccgatatcaaccaagaacgcggaatcaacgaaagctacaaaaagaatcttatggctctcaagaaatttgttatggtgaaattccttaatgactctatagtagatcctgtcgattcagaatggttcgggttctacaggtctggccaggcgaaggagactattcccctccaagaaacgtctctctatacacaagacagactcggactgaaagagatggataatgcgggccagttggtcttcttggctacggaaggcgatcatctccaactctccgaagagtggttctatgcccatataatcccgttcctgggctaa

SEQ ID NO: 17 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 18 - protein, 67 aa, synthetic construct
  REGION 1..67: note = source = /note="Description of Artificial Sequence: Synthetic polypeptide"
AYRPSETLCGGELVDTLQFVCGDRGSYFSRPASRVSRRSRGIVEECCFRSCDLALLETYCATPAKSE

SEQ ID NO: 19 - protein, 67 aa, synthetic construct
  REGION 1..67: note = source = /note="Description of Artificial Sequence: Synthetic polypeptide"
AYRPSETLCGGELVDTLQFVCGDRGFLFSRPASRVSRRSRGIVEECCFRSCDLALLETYCATPAKSE

SEQ ID NO: 20 - protein, 67 aa, synthetic construct
  REGION 1..67: note = source = /note="Description of Artificial Sequence: Synthetic polypeptide"
AYRPSETLCGGELVDTLQFVCGDRGFYFSRPASRVSRRSRGILEECCFRSCDLALLETYCATPAKSE

SEQ ID NO: 21 - protein, 67 aa, synthetic construct
  REGION 1..67: note = source = /note="Description of Artificial Sequence: Synthetic polypeptide"
AYRPSETLCGGELVDTLQFVCGDRGFYFSRPASRVSRRSRGIVEECCTRSCDLALLETYCATPAKSE

SEQ ID NO: 22 - protein, 67 aa, synthetic construct
  REGION 1..67: note = source = /note="Description of Artificial Sequence: Synthetic polypeptide"
AYRPSETLCGGELVDTLQFVCGDRGFYFSRPASRVSRRSRGIVEECCFSSCDLALLETYCATPAKSE

SEQ ID NO: 23 - protein, 67 aa, synthetic construct
  REGION 1..67: note = source = /note="Description of Artificial Sequence: Synthetic polypeptide"
AYRPSETLCGGELVDTLQFVCGDRGFYFSRPASRVSRRSRGIVEECCFRICDLALLETYCATPAKSE

SEQ ID NO: 24 - protein, 67 aa, synthetic construct
  REGION 1..67: note = source = /note="Description of Artificial Sequence: Synthetic polypeptide"
AYRPSETLCGGELVDTLQFVCGDRGFYFSRPASRVSRRSRGIVEECCFRSCDLRLLETYCATPAKSE

SEQ ID NO: 25 - protein, 67 aa, synthetic construct
  REGION 1..67: note = source = /note="Description of Artificial Sequence: Synthetic polypeptide"
AYRPSETLCGGELVDTLQFVCGDRGFYFSRPASRVSRRSRGIVEECCFRSCDLARLETYCATPAKSE

SEQ ID NO: 26 - protein, 67 aa, synthetic construct
  REGION 1..67: note = source = /note="Description of Artificial Sequence: Synthetic polypeptide"
AYRPSETLCGGELVDTLQFVCGDRGSLFSRPASRVSRRSRGILEECCTSICDLRRLETYCATPAKSE

SEQ ID NO: 27 - protein, 61 aa, synthetic construct
  REGION 1..61: note = source = /note="Description of Artificial Sequence: Synthetic polypeptide"
TLCGGELVDTLQFVCGDRGFLFSRPASRVSRRSRGIVEECCFRSCDLALLETYCATPARSE

SEQ ID NO: 28 - RNA, 274 nt, Cricket paralysis virus
  source 1..274: mol_type = other RNA
cggtgtcgaagtagaatttctatctcgacacgcggccttccaagcagttagggaaaccgacttctttgaagaagaaagctgactatgtgatcttattaaaattaggttaaatttcgaggttaaaaatagttttaatattgctatagtcttagaggtcttgtatatttatacttaccacacaagatggaccggagcagccctccaatatctagtgtaccctcgtgctcgctcaaacattaagtggtgttgtgcgaaaagaatctcacttcaagaa

SEQ ID NO: 29 - protein, 18 aa, Homo sapiens
MKLSLVAAMLLLLSAARA

SEQ ID NO: 30 - protein, 28 aa, synthetic construct
  REGION 1..28: note = source = /note="Description of Artificial Sequence: Synthetic peptide"
MKLSLVAAMLLLLSLVAAMLLLLSAARA

SEQ ID NO: 31 - protein, 25 aa, synthetic construct
  REGION 1..25: note = source = /note="Description of Artificial Sequence: Synthetic peptide"
MKLSLVAAMLLLLWVALLLLSAARA

SEQ ID NO: 32 - protein, 26 aa, synthetic construct
  REGION 1..26: note = source = /note="Description of Artificial Sequence: Synthetic peptide"
MKLSLVAAMLLLLSLVALLLLSAARA

SEQ ID NO: 33 - protein, 26 aa, synthetic construct
  REGION 1..26: note = source = /note="Description of Artificial Sequence: Synthetic peptide"
MKLSLVAAMLLLLALVALLLLSAARA

SEQ ID NO: 34 - RNA, 10 nt, synthetic construct
  misc_feature 1..10: note = source = /note="Description of Artificial Sequence: Synthetic oligonucleotide"
  source 1..10: mol_type = other RNA
gccrccatgg